SEQUENCE LISTING (42 sequences)

SEQ ID NO: 1 - DNA, 692 bp, Homo sapiens
  ref <308>: DATABASE ACCESSION NUMBER: GenBank/Acc. No. X14440
gtaatttttctctttactaattttgaccattgtttgcgttaacaatgccctgggctctgtaaagaatagtgtgttgattctttatcccagatgtttctcaagtggtcctgattttacagttcctaccaccagcttcccagtttaagctctgatggttggcctcaagcctgtgtcgtcccagcagcctcccgcctggccactctgactcagtctgtcctcctaaatatggccgtaagcttacccatcatgaaccactactcagggaggctccatgatagggcaaaaagtaaactctgaccagcttggttctaacccagctagtaaaatgtaaggattaggtaagatgttatttaaaactctttccagctcaaaaaactcctgattctaagatagtcacactctatgtgtgtctcttgcttgcctctgctgaaatattagtgactaagtggtataggagagactccgcagaacagcggaatgcatgagttttggacgtcgggtttgaggttctcctcaacctcttactaactttgtgattttgggcaaatcatttcctctttctggaaccctggtttcctcatctggagaaaggaaataattataataaccatatttcaaaatattgtttggagagtaatatagttaatgaatatgaaaagtgctttgtcaagtataatatgagcaaggttact

SEQ ID NO: 2 - DNA, 692 bp, Homo sapiens; identical to SEQ ID NO: 1
  ref <308>: DATABASE ACCESSION NUMBER: GenBank/Acc. No. M55418

SEQ ID NO: 3 - DNA, 692 bp, Chimpanzee
gtaatttttctctttactaattttgaccattgtttgcgttaacaatgccctgggctctgtaaagaatagtgtgttgattctttatcccagatgtttctcaagtggtcctgattttacagttcctaccaccagcttcccagtttaagctctgatggttggcctcaagcctgtgtcgtcccagcagcctcctgcctggccactctgactcagtctgtcctcctaaatatggccgtaagcttacccatcatgaaccactactcagggaggctccatgatagggcaaaaagtaaactctgaccagcttggttctaacccagctagtaaaatgtaaggattaggtaagatgttatttaaaactctttccagctcaaaaaactcctgattctaagatagtcacactctatgtgtgtctctcgtttgcctctgctgaaatattagtgactaagtggtataggagagactccgcagaacagcggaatgcatgagttttggacgtcgggtttgaggttctcctcaacctcctactaactttgtgattttgggcaaatcatttcctctttctggaaccctggtttcctcatctggagaaaggaaataattataataaccatatttcaaaatattgtttggagagtaatatagttaatgaatatgaaaagtgctttgtcaagtataatatgagcaaggttact

SEQ ID NO: 4 - DNA, 695 bp, Saimiri boliviensis
  ref <308>: DATABASE ACCESSION NUMBER: GenBank/Acc. No. U88981
gtaatttttctctttactaattttgaccattgtttgcgttaacagtgccctgggctctgtaaagaatagtgtgttgattcttcatcccagatgtttctcaagtggccctgattttacagtttctaccaccagcttcccagtttaagctcttgacggttggcctcaagcctgtgttgtcccagcaccctcctgcctggccactctgactcagtctctcctcctaaatatgactgtgaccttacccatcatgaaccactgctcagggaggctctatggtagggcaaaaagtcaactctgactgaccagcttggttctatcccagctagtaaaatgtaaggattaggtaagatgttatttaagattctttccagctcaacaaacttctgattctaagatagtcacactgtacatgtgtctctcatttggctctgctgaaatattattgactaagtggtatatgagagattctgcagaacaatgaaatgcatgacgttttgggcatcaggtttgaggttctcctcaatctcttactaactgtgtgactttgggcaattcatttcctttttctggaaccctggtttcctcatctggagaaaggaaatgattataataatatttcaaaatattgtttggagagtaatataattaacagatatgaaaagtgctttgtcaactataatatgagcaaagttact

SEQ ID NO: 5 - DNA, 690 bp, Baboon
gtaatttttctctttactaattttgaccattgtttgcgttaacaatgccctgggctctgtaaagaatagtgtgttgattctttatcccagatgtttctcaagtggtcctgattttacagttcctaccaccagcttcccagtttaagctctgatggttggcctcaagcctgtgtcgtcccagcagcctcctgcctggccactctgactcagtctctcctcctaaatatggccgtaagcttacccatcatgaaccaccagtgagggaggctccatgatagggcaaaaagtcaactctgaccagcttggttctatcccagctagtaaaatgtaaggattaggtaagatgttatttaagactatttccagctcaaaaactcctgattctaagatagtcacactctatgtgtgtctctcatttggctctgctgaaatattagtgactaagtggtataggagagactctgcagaacaatggaatgcatgagttttggacattgggtttgaggttctcctcaacctcttactaactgtgtgactttgggcaaatcatttctctttctggaaccctggtttcctcatgtggagaaaggaaataattataataaccatatatcaaaatattgtttggagaataatatacttaatgaatatgaaaagtactttgtcaagtataatatgagcaagtttact

SEQ ID NO: 6 - DNA, 692 bp, Orangutan
gtaatttttctctttactaattttgaccattgtttgcgttaacaatgccctgggctctgtaaagaatagtgtgttgattctttatcccagatgtttctcaagcggtcctgattttacagttcctaccaccagcttccctgtttaagctctgatggttggcctcaagcctgtgtcgtcccagcagcctcccgcctggctactctgactcagtctgtcctcctaaatatggccgtaagcttacccatcatgaaccactactcagggaggctccatgatagggcaaaaagtaaactctgaccagcttggttctatcccagctaataaaacgtaaggattaggtaagatgttatttaagactctttccagctcaaaaaactcctgattctaagatagtcacactctatgtgtgtctctcatttgcctctgctgaaatattagtgactaagtggtataggagagactccgcataacagtggaatgcatgagttttggacattgggtttgaggttctcctcaacctcttactaactgtatgactttgggcaaatcatttcctctttctggaaccctggtttcctcatctggagaaaggaaataattataataaccatatttcaaaatattgtttggagagtaatatagttaatgaatatgaaaagtgctttgtcaagtataatatgagcaaggttact

SEQ ID NO: 7 - DNA, 24 bp, Artificial Sequence
  Description of Artificial Sequence: Oligonucleotide primer “AMEL-A”
  ref <301>: AUTHORS: Sullivan, K. M. Mannucci, A. Kimpton, C. P. Gill, P.
  ref <302>: TITLE: A Rapid and Quantitative DNA Sex Test: Fluorescence-Based PCR Analysis of X-Y Homologous Gene Amelogenin
  ref <303>: JOURNAL: BioTechniques
  ref <304>: VOLUME: 15
  ref <305>: ISSUE: 4
  ref <306>: PAGES: 636-638
  ref <307>: DATE: OCT-1993
  ref <301>: AUTHORS: Sullivan, K. M. Mannucci, A. Kimpton, C. P. Gill, P.
  ref <302>: TITLE: A Rapid and Quantitative DNA Sex Test: Fluorescence-Based PCR Analysis of X-Y Homologous Gene Amelogenin
  ref <303>: JOURNAL: BioTechniques
  ref <304>: VOLUME: 15
  ref <305>: ISSUE: 4
  ref <306>: PAGES: 640-641
  ref <307>: DATE: OCT-1993
ccctgggctctgtaaagaatagtg

SEQ ID NO: 8 - DNA, 24 bp, Artificial Sequence
  Description of Artificial Sequence:  Complement of the oligonucleotide primer “AMEL-B”
  ref <301>: AUTHORS: Sullivan, K. M. Mannucci, A. Kimpton, C. P. Gill, P.
  ref <302>: TITLE: A Rapid and Quantitative DNA Sex Test: Fluorescence-Based PCR Analysis of X-Y Homologous Gene Amelogenin
  ref <303>: JOURNAL: BioTechniques
  ref <304>: VOLUME: 15
  ref <305>: ISSUE: 4
  ref <306>: PAGES: 636-638
  ref <307>: DATE: OCT-1993
  ref <301>: AUTHORS: Sullivan, K. M. Mannucci, A. Kimpton, C. P. Gill, P.
  ref <302>: TITLE: A Rapid and Quantitative DNA Sex Test: Fluorescence-Based PCR Analysis of X-Y Homologous Gene Amelogenin
  ref <303>: JOURNAL: BioTechniques
  ref <304>: VOLUME: 15
  ref <305>: ISSUE: 4
  ref <306>: PAGES: 640-641
  ref <307>: DATE: OCT-1993
cagcttcccagtttaagctctgat

SEQ ID NO: 9 - DNA, 20 bp, Artificial Sequence
  Description of Artificial Sequence: Oligonucleotide primer “DiFiore AMEL-F1”
accaccagcttcccagttta

SEQ ID NO: 10 - DNA, 20 bp, Artificial Sequence
  Description of Artificial Sequence:  Complement of oligonucleotide primer “DiFiore AMEL-R1”
  unsure (14): n at position 14 is a or t
cagcttggttctancccagc

SEQ ID NO: 11 - DNA, 20 bp, Artificial Sequence
  Description of Artificial Sequence: Oligonucleotide primer “DiFiore AMEL-R1”
  unsure (7): n at position 7 is a or t
gctgggntagaaccaagctg

SEQ ID NO: 12 - DNA, 174 bp, Pan troglodytes
  ref <308>: DATABASE ACCESSION NUMBER: GenBank/Acc. No. AF008917
agggggtaggctggttgggaggggttgagggggtgttgagggcggagaaatgaaagtttcattacgaaagttaacgtaacaaagaacctggtagaagtgagttttggatagtaaaataagtttcgaactctggcacctttaaattttgtcgcaccctccttgtttttgacaatg

SEQ ID NO: 13 - DNA, 476 bp, Pan troglodytes
  ref <308>: DATABASE ACCESSION NUMBER: GenBank/Acc. No. AJ222687
agggggtaggctggttgggaggggttgagggggtgttgagggcggagaaatgaaagtttcattacaaaagttaacgtaacaaagaacctggtagaagtgagttttggatagtaaaataagtttcgaactctggcacctttaaattttgtcgcaccctccttgtttttgacaatgcaatcatatgcttctgctatgttaagcgtattcaacagcgatgattacagtccagctgtgcaacagaatattcccgctctccggagaagctcttccttcctttgcactgaaagctataactctaagtatcagcgtgaaacgggagaaaacagtaaagacagcgtccaggatagagtgaagcgacccatgaacgcattcttcgtgtggtctcgcgatcagaggcgcaagatggctctagagaatcccagaatgcgaaactcagagatcagcaagcagctgggataccagtggaaaatgcttac

SEQ ID NO: 14 - DNA, 493 bp, Pan troglodytes
  ref <308>: DATABASE ACCESSION NUMBER: GenBank/Acc. No. X86380
atgcaatcatatgcttctgctatgttaagcgtattcaacagcgatgattacagtccagctgtgcaacagaatattcccgctctccggagaagctcttccttcctttgcactgaaagctataactctaagtatcagcgtgaaacgggagaaaacagtaaagatagcgtccaggatagagtgaagcgacccatgaacgcattcttcgtgtggtctcgcgatcagaggcgcaagatggctctagagaatcccagaatgcgaaactcagagatcagcaagcagctgggataccagtggaaaatgcttactgaagccgaaaaatggccattcttccaggaggcacagaaattacaggccatgcacagagagaaatacccgaattataagtatcgacctcgtcggaaggcgaacatgctgccgaagaattgcagtttgcttcccgcagatcccgcttcggtactctgcagcgaagtgcaactggacaacaggttgtaca

SEQ ID NO: 15 - DNA, 485 bp, Gorilla gorilla
  ref <308>: DATABASE ACCESSION NUMBER: GenBank/Acc. No. AJ003068
agggggtaggctggttgggcggggttgacggggtgttgagggcggagaaatgaaagtttcattacaaaagttaacgtaacaaagaatctggtagaagtgagttttggatagtaaaataagtttcgaactctggcacctttcaattttgtcgcaccctccttgtttttgacaatgcaatcatacgcttctgctatgttaagcgtattcaacagcgatgattacagtccagctgtgcaacagactattcccgctcaccggagaagctcttccttcctttgcactgaaagctgtaactctaagtatcagtgtgaaacgggagaaaacagtaaaggcagcgtccaggatcgagtgaagcgacccatgaacgcattcatcgtgtggtctcgcgatcagaggcgcaagatggctctagagaatcccagaatgcgaaactcagagatcagcaagcagctgggataccagtggaaaatgcttacagaagccga

SEQ ID NO: 16 - DNA, 493 bp, Gorilla gorilla
  ref <308>: DATABASE ACCESSION NUMBER: GenBank/Acc. No. X86382
atgcaatcatacgcttctgctatgttaagcgtattcaacagcgatgattacagtccagctgtgcaacagactattcccgctcaccggagaagctcttccttcctttgcactgaaagctgtaactctaagtatcagtgtgaaacgggagaaaacagtaaaggcagcgtccaggatcgagtgaagcgacccatgaacgcattcatcgtgtggtctcgcgatcagaggcgcaagatggctctagagaatcccagaatgcgaaactcagagatcagcaagcagctgggataccagtggaaaatgcttactgaagccgaaaaatggccattcttccaggaggcacagaaattacaggccatgcacagagagaaatacccgaattataagtatcgacctcgtcggaaggcgaagatgctgccgaagaattgcagtttgcttcccgcagatcccgcttcggtactctgcagcgaagtgcaactggacaaccggttgtaca

SEQ ID NO: 17 - DNA, 665 bp, Presbytis
agggggtaacctggttgggcggagttgagaggggtgttgggggcggagaaatgaaagttgcattacaaaagttaaggtaacaaagaatctggtggaagtaagttttggatagttaattaagtttccaaatctggcacctttcagctttgtcgcaccctccttgtttttgacaatgcaatcatatgcttctgctatgttaagcgtatttaacactgatgattacagtccagctgcgcaacagaatgttcctgctctccggagaagctcttccttcatttgcactgaaagctgtagctctaagtatcagtgtgaagcaggagaaaacagtaaaggcagcgtccaggatagagtgaagcgacccatgaacgcattcattgtgtggtctcgcgatcagaggcgcaagatggctctagagaatcccaaaatgcgaaactcggagatcagcaagcagctgggataccagtggaaaatgcttaccgaagccgataaatggccattcttccaggaggcacagaaactacaggccatgcatagagagaaatacccgaattataagtatcgacctcgtcggaaggcgaagatgctgcaaaacagttgcagtttgcttcccgcagatccttcttcggtactctgcagagaaatggaactggacaacaggttgtaca

SEQ ID NO: 18 - DNA, 665 bp, Trachypithecus
agggggttagctggttgggcggagttgagaggggtgttgggggcggagaaatgaaagtttcattacaaaagttaaggtaacaaagaatctggtagaagtaagttttggatagttaattaagtttccaaatctgacacctttcagttttgtcgcatcctccttgtttttgacaatgcaatcatatgcttctgctatgttaagcgtatttaacactgatgattacagtccagctgcgcaacagaatgttcctgctctccggagaagctcttccttcatttgcactgaaagctgtagctctaagtatcagtgtgaagcaggagaaaacagtaaaggcagcgtccaggatagagtgaagcgacccatgaacgcattcattgtgtggtctcgcgatcagaggcgcaagatggctctagagaatcccaaaatgcgaaactcagagatcagcaagcagctgggataccagtggaaaacgcttaccgaagccgataaatggccattcttccaggaggcacagaaactgcaggccatgcatagagagaaatacccgaattataagtatcgacctcgtcggaaggcgaagatgctgcaaaacagttgcagtttgcttccagcagatccttcttcggtactctgcagagaaatggaactggacaacagattgtaca

SEQ ID NO: 19 - DNA, 664 bp, Homo sapiens
  ref <308>: DATABASE ACCESSION NUMBER: GenBank/Acc. No. L08063
agggggtaggctggttgggcggggttgagggggtgttgagggcggagaaatgcaagtttcattacaaaagttaacgtaacaaagaatctggtagaagtgagttttggatagtaaaataagtttcgaactctggcacctttcaattttgtcgcactctccttgtttttgacaatgcaatcatatgcttctgctatgttaagcgtattcaacagcgatgattacagtccagctgtgcaagagaatattcccgctctccggagaagctcttccttcctttgcactgaaagctgtaactctaagtatcagtgtgaaacgggagaaaacagtaaaggcaacgtccaggatagagtgaagcgacccatgaacgcattcatcgtgtggtctcgcgatcagaggcgcaagatggctctagagaatcccagaatgcgaaactcagagatcagcaagcagctgggataccagtggaaaatgcttactgaagccgaaaaatggccattcttccaggaggcacagaaattacaggccatgcacagagagaaatacccgaattataagtatcgacctcgtcggaaggcgaagatgctgccgaagaattgcagtttgcttcccgcagatcccgcttcggtactctgcagcgaagtgcaactggacaacaggttgtaca

SEQ ID NO: 20 - DNA, 664 bp, Homo sapiens; identical to SEQ ID NO: 19
  ref <308>: DATABASE ACCESSION NUMBER: GenBank/Acc. No. L10102

SEQ ID NO: 21 - DNA, 664 bp, Homo sapiens; identical to SEQ ID NO: 19
  ref <308>: DATABASE ACCESSION NUMBER: GenBank/Acc. No. X53772

SEQ ID NO: 22 - DNA, 589 bp, Homo sapiens
  ref <308>: DATABASE ACCESSION NUMBER: GenBank/Acc. No. L10101
gtaacaaagaatctggtagaagtgagttttggatagtaaaataagtttcgaactctggcacctttcaattttgtcgcactctccttgtttttgacaatgcaatcatatgcttctgctatgttaagcgtattcaacagcgatgattacagtccagctgtgcaagagaatattcccgctctccggagaagctcttccttcctttgcactgaaagctgtaactctaagtatcagtgtgaaacgggagaaaacagtaaaggcaacgtccaggatagagtgaagcgacccatgaacgcattcatcgtgtggtctcgcgatcagaggcgcaagatggctctagagaatcccagaatgcgaaactcagagatcagcaagcagctgggataccagtggaaaatgcttactgaagccgaaaaatggccattcttccaggaggcacagaaattacaggccatgcacagagagaaatacccgaattataagtatcgacctcgtcggaaggcgaagatgctgccgaagaattgcagtttgcttcccgcagatcccgcttcggtactctgcagcgaagtgcaactggacaacaggttgtaca

SEQ ID NO: 23 - DNA, 665 bp, Cebus
tggaggtaggcaggttgggcggggttgagaggggtgctgggggcggagaaaagtaagttgcattacaaaagttaaggtaacaatgaatttggtagaagtaaatttgggatagtaaaatgagtttcccactctgtcatctttgaatttttcagtaccctgcttgtttttgacaatgcagtcttatgcttccactatgctgagagtatttaactgtgatgaatacagtccagctgcgcaacagaatatccctgcttccgggaaaagctcttccgtcgtttggactgagaactctagctcaaagtatcagtgcgaaacaggagaaaacagtaaaggcaacgtccagaagagagtgaagcgacccatgaacgctttcattgtgtggtctcgtgaccaaaggcgcaagatggctgtagagaatccccagatgcgaaactcagagatcagcaagcggctgggataccagtggaaattgcttactgaagccgaaaaatggccattcttccaggaggcacagaaactacaggccatgcacagagagaaatacccgaattataagtatcgacctcgtcggaaggccaacttactgcagaacaatgacagtttgcgtcccgtcgatccctcttcagagctctgtaacgaaatgcaagtagaagacaggttgtaca

SEQ ID NO: 24 - DNA, 493 bp, Pan paniscus
  ref <308>: DATABASE ACCESSION NUMBER: GenBank/Acc. No. X86381
atgcaatcatatgcttctgctatgttaagcgtattcaacagcgatgattacagtccaggtgtgcaacagaatattcccgctctccggagaagctcttccttcctttgcactgaaagctataactctaagtatcagcgtgaaacgggagaaaacagtaaagacagcgtccaggatagagtgaagcgacccatgaacgcattcttcgtgtggtctcgcgatcagaggcgcaagatggctctagagaatcccagaatgcgaaactcagagatcagcaagcagctgggataccagtggaaaatgcttactgcagccgaaaaatggccattcttccaggaggcacagaaattacaggccatgcacagagagaaatacccgaattataagtatcgacctcgtcggaaggcgaacatgctgccgaagaattgcagtttgcttcccgcagatcccgcttcggtactctgcagcgaagtgcaactggacaacaggttgtaca

SEQ ID NO: 25 - DNA, 496 bp, Pongo pygmaeus
  ref <308>: DATABASE ACCESSION NUMBER: GenBank/Acc. No. X86383
atgcaatcatatgcttctgctatgttaagcgtattcaacagtgatgattacagtccagctgtgcaacagaatattcccgctctccggagaagctcttccttcatttgcactgaaagctataactctaagtatcagtgtgaaacgggagaaaacagtaaaggcagcgtccaggatagagtgaagcgacccatgaacgcattcatcgtgtggtctcgcgatcagaggcgcaagatggctctagagaatcccaaaatgcgaaactcagagatcagcaagcagctgggataccagtggaaaatgcttactgaagccgaaaaatggccattcttccaggaggcacagaaactacaggccatgcatagagagaaatacccgaattataagtatcgacctcgtcggaaggcgaagatgctgcagaagagttgcagttcgcttcccgcagatcccgcttcggtactctgcagcgaagtgctgcaactggacaacaggttgtaca

SEQ ID NO: 26 - DNA, 493 bp, Callithrix jacchus
  ref <308>: DATABASE ACCESSION NUMBER: GenBank/Acc. No. X86386
atgcagtcttatgcttccgctatgttgagagtatttaacagtgatgaatacaatccagctgcgctacagaatatccctgattccgggaaaagctcttccgtcatttggactgacaactctagctcaaaggatcagtggcaaacaggagaaaacagtaaaggcagcgtccagaacagagtgaagcgacccatgaacgctttcattgtgtggtctcgtgatcaaaggcgcaagatggctgtagagaatccccaaatgcgaaattcagagatcagcaagcggctgggataccagtggaaattgcttactgaagccgaaaaatggccattcttccaggaggcacagaaactacaggccatgcacagagagaaatacccgaattataagtatcgacctcgtcggaaggccaatatgctgcagaacaatgacagtttgcttaccgccgatccatcttcagaactctgtggtgaaatgcaagcagaggacaggttgttca

SEQ ID NO: 27 - DNA, 490 bp, Papio hamadrayas
  ref <308>: DATABASE ACCESSION NUMBER: GenBank/Acc. No. X86385
atgcaatcatatgcttctaccatgttaagcgtatttaacactgatggttacagtccagctgcgcaacagaatattcctgctcctcggagaagctcttccttcatttgcactgaaagctgtagctctaagtatcagtgtgaagcaggagaaaacagtaaaggcagcgtccaggatagagtgaagcgacccatgaacgcattcattgtgtggtctcgcgatcagaggcgcaagatggctctagagaatcccaaaatgcgaaactcagagatcagcaagcagctgggataccagtggaaaatgcttaccgaagccgataaatggccattcttccaggaggcacagaaactacaggccatgcatagagagaaatacccgaattataagtatcgacctcgtcggaaggcgaagatgctgcaaaacagttgcagtttgcttccggcagatccctcttcggtaccctgcagagaagtgtacaacaacaggttgtaca

SEQ ID NO: 28 - DNA, 493 bp, Hylobates lar
  ref <308>: DATABASE ACCESSION NUMBER: GenBank/Acc. No. X86384
atgcaatcatatgcttctgctatgttaagcgtattcaacagtgattattacagtccagctgtgcaacagaatactcccgctctccggagaagctcttccttcatttgcactgaaagctgtaactctaagtatccgtgtgaaacgggagaaaacagtaaagtcagcgtccaggatagagtgaagcgacccatgaacgcattcatcgtgtggtctcgcgatcagaggcgcaagatggctctagagaatcccaaaatgcgaaactcagaaatcagcaagcagctgggataccggtggaaaatgcttactgaagccgaaaaatggccattcttccaggaggcacagaaactacaggccatgcacagagagaaatacccaaattataagtatcgacctcgtcggaaggcgaagatgctgcagaagagttgcagtttgcttcctgcagattcggcttcggtactctgcagcaaagtgcaactggacaacaggttgtaca

SEQ ID NO: 29 - DNA, 166 bp, Macaca fascicularis
  ref <308>: DATABASE ACCESSION NUMBER: GenBank/Acc. No. Z26906
cattgtgtggtctcgcgatcagaggcgcaagatggatctagagaatcccaaaatgcgaaactcagagatcagcaagcagctgggataccagtggaaaatgcttaccgaagccgataaatggccattcttccaggaggcacagaaactacaggccatgcatagagag

SEQ ID NO: 30 - DNA, 22 bp, Artificial Sequence
  Description of Artificial Sequence: Oligonucleotide primer “F11”
  ref <301>: AUTHORS: Santos, F. R. Pandya, A. Tyler-Smith, C.
  ref <302>: TITLE: Reliability of DNA-based Sex Tests
  ref <303>: JOURNAL: Nat. Genetics
  ref <304>: VOLUME: 18
  ref <305>: ISSUE: 2
  ref <306>: PAGES: 103-
  ref <307>: DATE: FEB-1998
ataagtatcgacctcgtcggaa

SEQ ID NO: 31 - DNA, 22 bp, Artificial Sequence
  Description of Artificial Sequence:  Complement of the oligonucleotide primer “R7”
  ref <301>: AUTHORS: Santos, F. R. Pandya, A. Tyler-Smith, C.
  ref <302>: TITLE: Reliability of DNA-based Sex Tests
  ref <303>: JOURNAL: Nat. Genetics
  ref <304>: VOLUME: 18
  ref <305>: ISSUE: 2
  ref <306>: PAGES: 103-
  ref <307>: DATE: FEB-1998
tcggtactctgcagcgaagtgc

SEQ ID NO: 32 - DNA, 20 bp, Artificial Sequence
  Description of Artificial Sequence: Oligonucleotide primer “DiFiore SRY-F1”
agtgaagcgacccatgaacg

SEQ ID NO: 33 - DNA, 20 bp, Artificial Sequence
  Description of Artificial Sequence:  Complement of the oligonucleotide primer “DiFiore SRY-R1”
ccattcttccaggaggcaca

SEQ ID NO: 34 - DNA, 20 bp, Artificial Sequence
  Description of Artificial Sequence: Oligonucleotide primer “DiFiore SRY-R1”
tgtgcctcctggaagaatgg

SEQ ID NO: 35 - DNA, 710 bp, Lemur
gtaatttttctttttactaattttgaccattgtttgggttaacaatgccctggagctctgtaaagaatagtgtgttgattcttcatccaagatgtttcttgaataatcccaattttacagttcttaccaccagttttccagtttaagccctgatgggtggcctcaagcctgcattgtcccagcaccctcctacctggccactctgagtctgtcctcctaaacatggctataattttatctatcatgaactaccacttagggaggctgcatggtagggtggaaagagaactctggctgaatagccttgttgtgtcccagccagtacaatgtggggattaggtaggatgttatctaagattttttccaagctcagaaactcctgattataagacatttacacttgatgtgagtctctcacttgacttcactgaaagatgagtgactaagtgctgtatgagggactgtgcagaggaatggaaagcaccagagctctgggtctcgggtttgaggttctcctcaatcccttactaactgcatgacttcgggaaaaccattttctctctctggaactttggttccctcagctggagaagggaaataattatgataagcatatttcaaaacattgtttggaaagtaaaataattattgaatatgaaaagtgttttgtcaagtataacttgagcaaagttaataattatttgttatat

SEQ ID NO: 36 - DNA, 701 bp, Otolemur
gtaatttttctctttactaactttgaccattatttgacttagcaatgccttggggctctgtaaagaatagtgtgttgattcttcattcagtgtatttctcaaataatcccaattttacagtcctaccaccagcttcctagtttaaaccctgaaggatggcctcaagcctgcattgatccagcatccttctacctggccactctcagcctctcctcctaaaaataatgttatctgttatgaacaaccacttagggaggctcccacactagggcagaaagagaattctggctgaacaaccttgctctgtcccagccagtagaatgtggggattaggtaagatgttatttaagtttttttccagctcaaaaaactcctgattctaagacattcacacttgatgttgtccctcacatggcttcactgaaagatgagtggctcagtgccacatgaggggctctgcagaggtatggaaaacatcagaattcccagtatcaggtttgaggtccttctcaatcccttgctaactacaggaccttgggaaaatcattttctctctctggaactttggctccctcatttggagaagaaaaatatgtatgataagcatagcatatttcaaaatattgcttggaaagtaatacagttattgactatgaaaagtgtttcatcaagtataacttgagaaaggttaacaattacat

SEQ ID NO: 37 - DNA, 17 bp, Artificial Sequence
  Description of Artificial Sequence: Oligonucleotide primer “DiFiore AMEL-F1 (strep)”
tggcctcaagcctgcat

SEQ ID NO: 38 - DNA, 22 bp, Artificial Sequence
  Description of Artificial Sequence:  Complement of the oligonucleotide primer “DiFiore AMEL-R1 (strep)”
  unsure (16): n at position 16 is c or t
tgtggggattaggtangatgtt

SEQ ID NO: 39 - DNA, 22 bp, Artificial Sequence
  Description of Artificial Sequence: Oligonucleotide primer “DiFiore AMEL-R1 (strep)”
  unsure (7): n at position 7 is c or t
aacatcntacctaatccccaca

SEQ ID NO: 40 - DNA, 668 bp, Artificial Sequence
  Description of Artificial Sequence:  Consensus sequence of the primate SRY gene
  unsure (1): n at position 1 is a or t
  unsure (4): n at position 4 is a or g
  unsure (8): n at position 8 is a or t
  unsure (9): n at position 9 is a or g
  unsure (10): n at position 10 is g or c
  unsure (12): n at position 12 is a or t
  unsure (20): n at position 20 is a or c
  unsure (23): n at position 23 is a or g
  unsure (29): n at position 29 is g or c
  unsure (30): n at position 30 is a or absent
  unsure (37): n at position 37 is t or c
  unsure (40): n at position 40 is a or g
  unsure (52): n at position 52 is a or t
  unsure (54): n at position 54 is a, t, or c
  unsure (60): n at position 60 is t or g
  unsure (67): n at position 67 is a or g
  unsure (76): n at position 76 is g or c
  unsure (84): n at position 84 is a or t
  unsure (88)..(89): n at positions 88 and 89 is t or c
  unsure (94): n at position 94 is a or g
  unsure (100): n at position 100 is a or g
  unsure (102): n at position 102 is a or g
  unsure (106): n at position 106 is t or g
  unsure (114): n at position 114 is a or t
  unsure (117): n at position 117 is a or t
  unsure (119): n at position 119 is a or g
  unsure (126): n at position 126 is g or c
  unsure (127): n at position 127 is a or c
  unsure (129): n at position 129 is a or c
  unsure (134): n at position 134 is a, t, or g
  unsure (137): n at position 137 is t or c
  unsure (142): n at position 142 is a, g, or c
  unsure (144): n at position 144 is a or g
  unsure (145): n at position 145 is t or c
  unsure (149): n at position 149 is t or g
  unsure (150): n at position 150 is t or c
  unsure (151): n at position 151 is a or c
  unsure (153): n at position 153 is t or c
  unsure (155)..(156): n at positions 155 and 156 is t or c
  unsure (159): n at position 159 is g or c
  unsure (178): n at position 178 is a or g
  unsure (181): n at position 181 is a or t
  unsure (184): n at position 184 is t or c
  unsure (190): n at position 190 is t or c
  unsure (191): n at position 191 is a or g
  unsure (193): n at position 193 is t or c
  unsure (197): n at position 197 is t or c
  unsure (199): n at position 199 is a or g
  unsure (202): n at position 202 is a or c
  unsure (208): n at position 208 is t or c
  unsure (212): n at position 212 is a or t
  unsure (213): n at position 213 is g or c
  unsure (214): n at position 214 is t or c
  unsure (218): n at position 218 is t or g
  unsure (219): n at position 219 is a or g
  unsure (220): n at position 220 is a or t
  unsure (225): n at position 225 is a or g
  unsure (231): n at position 231 is g or c
  unsure (234): n at position 234 is t or c
  unsure (237): n at position 237 is a or t
  unsure (239): n at position 239 is g or c
  unsure (243): n at position 243 is a or c
  unsure (245): n at position 245 is a or g
  unsure (246)..(247): n at positions 246 and 247 is t or c
  unsure (250): n at position 250 is t or c
  unsure (252): n at position 252 is a or c
  unsure (254): n at position 254 is t or c
  unsure (255): n at position 255 is a, t, or c
  unsure (256): n at position 256 is t or c
  unsure (257): n at position 257 is g or c
  unsure (261): n at position 261 is a or g
  unsure (272): n at position 272 is t or g
  unsure (275): n at position 275 is a, g, or c
  unsure (280): n at position 280 is g or c
  unsure (286): n at position 286 is a, g, or c
  unsure (288): n at position 288 is a or g
  unsure (291): n at position 291 is a, g, or c
  unsure (294): n at position 294 is a or g
  unsure (298): n at position 298 is a or t
  unsure (302): n at position 302 is t or g
  unsure (306): n at position 306 is a or c
  unsure (308): n at position 308 is t or c
  unsure (310): n at position 310 is t, g, or c
  unsure (311): n at position 311 is g or c
  unsure (314): n at position 314 is a or g
  unsure (316): n at position 316 is a or g
  unsure (333): n at position 333 is a, t, or g
  unsure (334): n at position 334 is t or c
  unsure (336): n at position 336 is a or g
  unsure (344): n at position 344 is a or g
  unsure (346): n at position 346 is t, g, or c
  unsure (347): n at position 347 is a or c
  unsure (370): n at position 370 is a or t
  unsure (374): n at position 374 is a or t
  unsure (376): n at position 376 is t or c
  unsure (388): n at position 388 is t or c
  unsure (391): n at position 391 is t or c
  unsure (394): n at position 394 is a or g
  unsure (408): n at position 408 is a or c
  unsure (410): n at position 410 is g or c
  unsure (422): n at position 422 is a or c
  unsure (423)..(424): n at positions 423 and 424 is a or g
  unsure (433): n at position 433 is t or c
  unsure (436): n at position 436 is a or g
  unsure (439): n at position 439 is a or g
  unsure (450): n at position 450 is a or g
  unsure (462): n at position 462 is a or g
  unsure (470): n at position 470 is a or t
  unsure (471): n at position 471 is t or c
  unsure (478): n at position 478 is a, t, or c
  unsure (480): n at position 480 is a or c
  unsure (487): n at position 487 is a or t
  unsure (518): n at position 518 is t or c
  unsure (520): n at position 520 is a or g
  unsure (532): n at position 532 is t or c
  unsure (547): n at position 547 is a or g
  unsure (577): n at position 577 is g or c
  unsure (580): n at position 580 is t, g, or c
  unsure (581): n at position 581 is a or t
  unsure (583): n at position 583 is a or g
  unsure (588): n at position 588 is a or c
  unsure (589): n at position 589 is a or g
  unsure (592): n at position 592 is g or c
  unsure (594): n at position 594 is a or g
  unsure (596): n at position 596 is t or g
  unsure (597): n at position 597 is a or g
  unsure (603): n at position 603 is t or c
  unsure (606): n at position 606 is t or g
  unsure (608): n at position 608 is a or c
  unsure (610): n at position 610 is any nucleotide
  unsure (612): n at position 612 is t or c
  unsure (613): n at position 613 is a or c
  unsure (617): n at position 617 is t or c
  unsure (619): n at position 619 is any nucleotide
  unsure (620): n at position 620 is t or g
  unsure (625): n at position 625 is a or g
  unsure (627): n at position 627 is a or t
  unsure (628): n at position 628 is a or g
  unsure (630): n at position 630 is t or c
  unsure (634): n at position 634 is t or c
  unsure (635)..(636): n at positions 635 and 636 is a or g
  unsure (637): n at position 637 is a, t, or c
  unsure (638): n at position 638 is a or g
  unsure (641): n at position 641 is a or g
  unsure (644): n at position 644 is t, c, or absent
  unsure (645): n at position 645 is t or absent
  unsure (646): n at position 646 is a, g, or absent
  unsure (647): n at position 647 is is g, c, or absent
  unsure (649): n at position 649 is a or absent
  unsure (650): n at position 650 is g, c, or absent
  unsure (651): n at position 651 is t, c, or absent
  unsure (652): n at position 652 is a, g, or absent
  unsure (653): n at position 653 is g or absent
  unsure (655): n at position 655 is a, g, or c
  unsure (656): n at position 656 is a or g
  unsure (659): n at position 659 is a or c
  unsure (661): n at position 661 is a or g
  unsure (666): n at position 666 is a or t
nggnggtnnncnggttgggnggngttgannggggtgntgngggcggagaaangnaagttncattacnaaagttaangtaacaangaanntggtngaagtnantttnggatagtnaantnagtttcnnantctgncanctttnanntttnnngnannctncttgtttttgacaatgcantcntangcttcnncnatgntnagngtattnaacnnngatnnntacantccagntgngcnanagantnnnccngntnnnngganaagctcttccntcntttgnactgananctntanctcnaagnatcngngnnaancnggagaaaacagtaaagnnancgtccagnanngagtgaagcgacccatgaacgcnttcntngtgtggtctcgngancanaggcgcaagatggntntagagaatcccnnnatgcgaaantcnganatcagcaagcngctgggataccngtggaaanngcttacngnagccganaaatggccattcttccaggaggcacagaaantncaggccatgcanagagagaaatacccnaattataagtatcgacctcgtcggaaggcnaanntnctgcnnaanantnncagttngcntncngnngatncnncttcngnncnctgnnnnnaantgnnnnannnnnannacngnttgtnca

SEQ ID NO: 41 - DNA, 698 bp, Artificial Sequence
  Description of Artificial Sequence:  Consensus sequence of the anthropoid amelogenin gene
  unsure (45): n at position 45 is a or g
  unsure (83): n at position 83 is t or c
  unsure (103): n at position 103 is t or c
  unsure (106): n at position 106 is t or c
  unsure (122): n at position 122 is t or c
  unsure (139): n at position 139 is a or t
  unsure (151): n at position 151 is t or absent
  unsure (154): n at position 154 is t or c
  unsure (175): n at position 175 is t or c
  unsure (185): n at position 185 is g or c
  unsure (191): n at position 191 is t or c
  unsure (199): n at position 199 is t or c
  unsure (215): n at position 215 is g or c
  unsure (230): n at position 230 is a or g
  unsure (232): n at position 232 is t or c
  unsure (235): n at position 235 is a or g
  unsure (237): n at position 237 is g or c
  unsure (257): n at position 257 is t or c
  unsure (258): n at position 258 is a or g
  unsure (259): n at position 259 is g or c
  unsure (261): n at position 261 is g or c
  unsure (272): n at position 272 is t or c
  unsure (276): n at position 276 is a or g
  unsure (290): n at position 290 is a or c
  unsure (300): n at position 300 is t or absent
  unsure (301): n at position 301 is g or absent
  unsure (302): n at position 302 is a or absent
  unsure (303): n at position 303 is c or absent
  unsure (317): n at position 317 is a or t
  unsure (326): n at position 326 is a or g
  unsure (332): n at position 332 is t or c
  unsure (360): n at position 360 is a or g
  unsure (362): n at position 362 is t or c
  unsure (364): n at position 364 is a or c
  unsure (377): n at position 377 is a, c, or absent
  unsure (383): n at position 383 is t or c
  unsure (406): n at position 406 is g or c
  unsure (409): n at position 409 is t or c
  unsure (410): n at position 410 is a or g
  unsure (420): n at position 420 is t or c
  unsure (421): n at position 421 is a or g
  unsure (422): n at position 422 is t or c
  unsure (426): n at position 426 is g or c
  unsure (443): n at position 443 is t or g
  unsure (459): n at position 459 is t or g
  unsure (466): n at position 466 is t or c
  unsure (469): n at position 469 is t or c
  unsure (473): n at position 473 is t or g
  unsure (478): n at position 478 is a or g
  unsure (479): n at position 479 is t or c
  unsure (481): n at position 481 is a or g
  unsure (491): n at position 491 is c or absent
  unsure (499): n at position 499 is a or g
  unsure (501): n at position 501 is a or g
  unsure (503): n at position 503 is t or c
  unsure (504): n at position 504 is a or g
  unsure (524): n at position 524 is t or c
  unsure (528): n at position 528 is t or c
  unsure (537): n at position 537 is t or g
  unsure (539): n at position 539 is a or g
  unsure (543): n at position 543 is t or c
  unsure (553): n at position 553 is a or t
  unsure (560): n at position 560 is c or absent
  unsure (563): n at position 563 is t or c
  unsure (588): n at position 588 is g or c
  unsure (603): n at position 603 is a or g
  unsure (612): n at position 612 is a or absent
  unsure (614)..(615): n at positions 614 and 615 is c or absent
  unsure (620): n at position 620 is a or t
  unsure (640): n at position 640 is a or g
  unsure (648): n at position 648 is a, g, or c
  unsure (653): n at position 653 is t or c
  unsure (654)..(655): n at positions 654 and 655 is a or g
  unsure (667): n at position 667 is a or g
  unsure (677): n at position 677 is g or c
  unsure (692): n at position 692 is a or g
  unsure (693): n at position 693 is t or g
gtaatttttctctttactaattttgaccattgtttgcgttaacantgccctgggctctgtaaagaatagtgtgttgattcttnatcccagatgtttctcaagnggncctgattttacagttnctaccaccagcttcccngtttaagctctnganggttggcctcaagcctgtgtngtcccagcancctccngcctggcnactctgactcagtctntcctcctaaatatgncngtnancttacccatcatgaaccacnnntnagggaggctcnatgntagggcaaaaagtnaactctgacnnnncagcttggttctancccagctantaaaangtaaggattaggtaagatgttatttaanantntttccagctcaanaaactnctgattctaagatagtcacactntanntgtgtctctnnnttgnctctgctgaaatattantgactaagtggtatangagagantcngcanaacanngnaatgcatgangttttggncntnnggtttgaggttctcctcaanctcntactaactntntgantttgggcaantcatttnctntttctggaaccctggtttcctcatntggagaaaggaaatnattataatnannatatntcaaaatattgtttggagantaatatanttaannnatatgaaaagtnctttgtcaantataatatgagcaannttact

SEQ ID NO: 42 - DNA, 717 bp, Artificial Sequence
  Description of Artificial Sequence:  Consensus sequence of the strepsirhine amelogenin gene
  unsure (12): n at position 12 is t or c
  unsure (21): n at position 21 is t or c
  unsure (32): n at position 32 is a or g
  unsure (37): n at position 37 is a or g
  unsure (38): n at position 38 is g or c
  unsure (42): n at position 42 is a or g
  unsure (50): n at position 50 is t or c
  unsure (54): n at position 54 is a or g
  unsure (87): n at position 87 is t or c
  unsure (90): n at position 90 is a or g
  unsure (91): n at position 91 is t or g
  unsure (92): n at position 92 is a or g
  unsure (94): n at position 94 is a or g
  unsure (100): n at position 100 is t or c
  unsure (101): n at position 101 is a or g
  unsure (121): n at position 121 is t or absent
  unsure (124): n at position 124 is t or c
  unsure (134): n at position 134 is t or c
  unsure (137): n at position 137 is t or c
  unsure (139): n at position 139 is t or c
  unsure (147): n at position 147 is a or g
  unsure (154): n at position 154 is a or t
  unsure (157): n at position 157 is a or g
  unsure (177): n at position 177 is a or t
  unsure (178): n at position 178 is t or c
  unsure (185): n at position 185 is t or c
  unsure (189): n at position 189 is t or c
  unsure (205): n at position 205 is g or c
  unsure (208): n at position 208 is t or c
  unsure (211): n at position 211 is g or c
  unsure (219): n at position 219 is a or absent
  unsure (222): n at position 222 is c or absent
  unsure (224): n at position 224 is t or absent
  unsure (225)..(226): n at positions 225 and 226 is g or absent
  unsure (227): n at position 227 is a or c
  unsure (228): n at position 228 is t or absent
  unsure (234): n at position 234 is t or g
  unsure (241): n at position 241 is a or g
  unsure (243): n at position 243 is t or c
  unsure (250): n at position 250 is a or t
  unsure (267): n at position 267 is c or absent
  unsure (268): n at position 268 is g or c
  unsure (271): n at position 271 is t or c
  unsure (272): n at position 272 is a or g
  unsure (273): n at position 273 is g or c
  unsure (279): n at position 279 is t or c
  unsure (280): n at position 280 is a or g
  unsure (290): n at position 290 is t or c
  unsure (301): n at position 301 is t or c
  unsure (303): n at position 303 is a or g
  unsure (309): n at position 309 is t or c
  unsure (311): n at position 311 is g or c
  unsure (326): n at position 326 is g or c
  unsure (344): n at position 344 is a or g
  unsure (353): n at position 353 is t or c
  unsure (358): n at position 358 is a or t
  unsure (367): n at position 367 is a or absent
  unsure (373): n at position 373 is a or absent
  unsure (375): n at position 375 is a or g
  unsure (388): n at position 388 is a or c
  unsure (398): n at position 398 is t or c
  unsure (410): n at position 410 is g or absent
  unsure (411): n at position 411 is a or t
  unsure (415): n at position 415 is t or c
  unsure (421): n at position 421 is a or t
  unsure (424): n at position 424 is a or g
  unsure (444): n at position 444 is a or g
  unsure (447): n at position 447 is a or c
  unsure (453): n at position 453 is t or c
  unsure (454): n at position 454 is a or g
  unsure (455): n at position 455 is t or c
  unsure (463): n at position 463 is a or g
  unsure (466): n at position 466 is g or c
  unsure (475): n at position 475 is a or t
  unsure (483): n at position 483 is a or g
  unsure (486): n at position 486 is t or c
  unsure (491): n at position 491 is a or g
  unsure (492): n at position 492 is t or c
  unsure (495): n at position 495 is t or c
  unsure (496): n at position 496 is g or c
  unsure (497): n at position 497 is a or g
  unsure (500): n at position 500 is a or c
  unsure (503): n at position 503 is a or g
  unsure (514): n at position 514 is t or c
  unsure (517): n at position 517 is t or c
  unsure (529): n at position 529 is a or g
  unsure (536): n at position 536 is a or g
  unsure (539): n at position 539 is t or g
  unsure (543): n at position 543 is t or c
  unsure (545): n at position 545 is t or c
  unsure (553): n at position 553 is t or c
  unsure (578): n at position 578 is t or c
  unsure (586): n at position 586 is t or g
  unsure (587): n at position 587 is t or c
  unsure (596)..(597): n at positions 596 and 597 is a or g
  unsure (603): n at position 603 is a or t
  unsure (604): n at position 604 is t or g
  unsure (611): n at position 611 is a or absent
  unsure (613): n at position 613 is g or absent
  unsure (614): n at position 614 is c or absent
  unsure (616): n at position 616 is t or absent
  unsure (617): n at position 617 is a or absent
  unsure (631): n at position 631 is t or c
  unsure (636): n at position 636 is t or c
  unsure (648): n at position 648 is a or t
  unsure (650): n at position 650 is t or c
  unsure (652): n at position 652 is a or g
  unsure (660): n at position 660 is a or c
  unsure (675): n at position 675 is t or c
  unsure (676): n at position 676 is a or g
  unsure (693): n at position 693 is a or c
  unsure (696): n at position 696 is a or g
  unsure (702): n at position 702 is t or absent
  unsure (703): n at position 703 is a or c
  unsure (705)..(706): n at positions 705 and 706 is t or absent
  unsure (708)..(709): n at positions 708 and 709 is t or absent
  unsure (711): n at position 711 is g or absent
  unsure (712): n at position 712 is t or absent
  unsure (715): n at position 715 is t or c
gtaatttttctntttactaantttgaccattntttgnnttancaatgccntggngctctgtaaagaatagtgtgttgattcttcatncannntntttctnnaataatcccaattttacagntcntaccaccagnttncnagtttaanccctganggntggcctcaagcctgcattgnnccagcancctnctacctggccactctnagnctntcctcctnaanannnnnataatnttatctntnatgaacnaccacttagggaggctnncannntagggnngaaagagaantctggctgaananccttgntntgtcccagccagtanaatgtggggattaggtangatgttatntaagnttttttccnagctcnanaaactcctgattntaagacattnacacttgatgtnngtcnctcacntgncttcactgaaagatgagtgnctnagtgcnnnatgagggnctntgcagaggnatggaaancancaganntcnnngtntcnggtttgaggtnctnctcaatcccttnctaactncangacntngggaaaancattttctctctctggaactttggntccctcanntggagaagnnaaatanntatgatnannanngcatatttcaaaanattgnttggaaagtaanananttattgantatgaaaagtgtttnntcaagtataacttgagnaangttaannannanntnntanat

(19) United States
(12) Patent Application Publication
(10) Pub. No.: US 2007/0048755 A1
(43) Pub. Date: Mar. 1, 2007

(54) Assay for determining the sex of primates

(75) Inventor: Anthony Di Fiore, New York, NY (US)

Correspondence Address: NIXON PEABODY LLP - PATENT GROUP, CLINTON SQUARE, P.O. BOX 31051, ROCHESTER, NY, 14603-1051, US

(73) Assignee: New York University, New York, NY (US)

(21) Appl. No.: 11/386,523

(22) Filed: Mar. 22, 2006

Related U.S. Application Data
(60) Provisional application No. 60/663,980, filed on Mar. 22, 2005.

(51) Int. Cl. C12Q 1/68

(52) U.S. Cl. 435/6

(57) ABSTRACT

The present invention relates to methods for identifying the sex of a primate by providing a biological sample collected from the primate and contacting the biological sample with one or more probes that hybridize to a target SRY nucleic acid molecule at a particular location within a consensus SRY nucleotide sequence. Any hybridization of the one or more probes at that location is detected, and the sex of the primate is identified based on whether any hybridization occurs. Oligonucleotide probes that hybridize to fragments of SRY or amelogenin are also disclosed.

This application claims the benefit of U.S. Provisional Patent Application Ser. No. 60/663,980, filed Mar. 22, 2005, which is hereby incorporated by reference in its entirety.

### FIELD OF THE INVENTION

This invention is directed generally to methods of determining the sex of a primate DNA sample and oligonucleotide probes useful for this determination.

### BACKGROUND OF THE INVENTION

The field of primate molecular ecology is growing rapidly and holds much promise for providing insights into aspects of primate social structure that are difficult to address in traditional observational studies (Di Fiore, “Molecular Genetic Approaches to the Study of Primate Behavior, Social Organization, and Reproduction,” Yrbk. Phys. Anthropol., 46:62-99 (2003)). A number of recent molecular studies have used samples collected either remotely or noninvasively from wild individuals to shed light on the mating systems, social behavior, dispersal patterns, population structure, and within-group patterns of relatedness of a range of nonhuman primates (chimpanzees: Morin et al., “Kin Selection, Social Structure, Gene Flow, and the Evolution of Chimpanzees,” Science, 265:1193-1201 (1994); Morin et al., “Paternity Exclusion in a Community of Wild Chimpanzees Using Hypervariable Simple Sequence Repeats,” Mol. Ecol., 3:469-478 (1994); Gagneux et al., “Female Reproductive Strategies, Paternity and Community Structure in Wild West African Chimpanzees,” Anim. Behav., 57:19-32 (1999); Mitani et al., “Male Affiliation, Cooperation and Kinship in Wild Chimpanzees,” Anim. Behav., 59:885-893 (2000); Vigilant et al., “Paternity and Relatedness in Wild Chimpanzee Communities,” Proc. Nat. Acad. Sci. USA, 98:12890-12895 (2001); bonobos: Gerloff et al., “Intracommunity Relationships, Dispersal Pattern, and Paternity Success in a Wild Living Community of Bonobos (Pan paniscus) Determined from DNA Analysis of Faecal Samples,” Proc. R. Soc. Lond. B., 266:1189-1195 (1999); Hohmann et al., “Social Bonds and Genetic Ties: Kinship, Association, and Affiliation in a Community of Bonobos (Pan paniscus),” Behaviour, 136:1219-1235 (1999); gorillas: Bradley et al., “Dispersed Male Networks in Western Gorillas,” Current Biol., 14:510-513 (2004); baboons: Smith et al., “Wild Female Baboons Bias Their Social Behaviour Towards Paternal Half-Sisters,” Proc. R. Soc. Lond. B., 270:503-510 (2003); leaf monkeys: Rosenblum et al., “High Mitochondrial DNA Diversity With Little Structure Within and Among Leaf Monkey Populations (Trachypithecus cristatus and Trachypithecus auratus),” Int. J. Primatol., 18:1005-1028 (1997); woolly monkeys: Di Fiore, “Molecular Perspectives on Dispersal in Lowland Woolly Monkeys (Lagothrix lagotricha poeppigii),” Am. J. Phys. Anthropol., S34:63 (2002)). Indeed, molecular ecological studies utilizing such samples may be the only tractable way for primate biologists and conservationist geneticists to gain critical information about taxa that are highly endangered, elusive, or difficult to habituate. Nonetheless, in order to take full advantage of these kinds of samples for investigating a number of fundamental aspects of primate social systems (e.g., the sex composition of groups, sex-biases in dispersal patterns), researchers must be able to confidently and reliably identify the sex of sampled individuals.

Several molecular methods have been developed for sex assignment in humans (e.g., Nakahori et al., “Sex Identification by Polymerase Chain Reaction Using X-Y Homologous Primers,” Am. J Med. Genet., 39:472-473 (1991); Iida et al., “Sex Identification by Polymerase Chain Reaction Using a Y-Autosome Homologous Primer Set,” Japanese J. of Human Genetics, 38:429-431 (1993); Sullivan et al., “A Rapid and Quantitative DNA Sex Test: Fluorescence-Based PCR Analysis of X-Y Homologous Gene Amelogenin,” BioTechniques, 15:637-641 (1993); Mannucci et al., “Forensic Application of a Rapid and Quantitative DNA Sex Test by Amplification of the X-Y Homologous Gene Amelogenin,” Int. J. Legal Med., 106:190-193 (1994); Reynolds & Varlaro, “Gender Determination of Forensic Samples Using PCR Amplification of ZFX/ZFY Gene Sequences,” J. Forensic Sci., 41:279-286 (1996); Stacks & Witte, “Sex Determination of Dried Blood Stains Using the Polymerase Chain Reaction (PCR) With Homologous X-Y Primers of the Zinc Finger Protein Gene,” J. Forensic Science, 41:287-290 (1996); Akane, “Sex Determination by PCR Analysis of the X-Y Amelogenin Gene,” Methods in Mol. Biol., 98:245-249 (1998)), but few have proven useful in other primates (but see Wilson & Erlandsson, “Sexing of Human and Other Primate DNA,” Biol. Chem., 379:1287-1288 (1998)). The most commonly used methods for sex typing human samples rely on the presence of fixed polymorphisms between the X- and Y-borne copies of the nuclear gene for the enamel protein amelogenin (Akane et al., “Sex Identification of Forensic Specimens by Polymerase Chain Reaction (PCR): Two Alternative Methods,” Forensic Sci. Int'l, 49:81-88 (1991); Akane et al., “Sex Determination of Forensic Samples by Dual PCR Amplification of an X-Y Homologous Gene,” Forensic Sci. Int'l, 52:143-148 (1992); Nakahori et al., “Sex Identification by Polymerase Chain Reaction Using X-Y Homologous Primers,” Am. J Med. Genet., 39:472-473 (1991); Sullivan et al., “A Rapid and Quantitative DNA Sex Test: Fluorescence-based PCR Analysis of X-Y Homologous Gene Amelogenin,” BioTechniques, 15:637-641 (1993); Mannucci et al., “Forensic Application of a Rapid and Quantitative DNA Sex Test by Amplification of the X-Y Homologous Gene Amelogenin,” Int. J. Legal Med., 106:190-193 (1994); Faerman et al., “Sex Identification of Archaeological Human Remains Based on Amplification of the X and Y Amelogenin Alleles,” Gene, 167:327-332 (1995); Haas-Rochholz & Weiler, “Additional Primer Sets for an Amelogenin Gene PCR-based DNA-sex Test,” Int. J. Legal Med., 110:312-315 (1997)). The amelogenin gene is located outside the recombining pseudoautosomal region on the short arm of both the X and Y chromosomes (Nakahori et al., “A Human X-Y Homologous Region Encodes ‘Amelogenin,’” Genomics, 9:264-269 (1991); Bailey et al., “The X-Y Homologous Amelogenin Maps to the Short Arms of Both the X and Y Chromosomes and is Highly Conserved in Primates,” Genomics, 14:203-205 (1992)). In humans, one intron of the gene copy on the X chromosome bears a fixed 6 base pair deletion, and, more than a decade ago, a simple PCR-based sex test was developed for humans in which a single primer pair is used to amplify short, homologous regions of the X and Y containing this sequence-length variation (Sullivan et al., “A Rapid and Quantitative DNA Sex Test: Fluorescence-based PCR Analysis of X-Y Homologous Gene Amelogenin,” BioTechniques, 15:637-641 (1993); Mannucci et al., “Forensic Application of a Rapid and Quantitative DNA Sex Test by Amplification of the X-Y Homologous Gene Amelogenin,” Int. J. Legal Med., 106:190-193 (1994)). Following separation and visualization, samples from male (XY) individuals show two PCR fragment bands, which differ in length by 6 base pairs, while samples from females (XX) manifest only the smaller amplicon.

Although a variant of this simple one-tube/one-reaction assay is now the standard used in forensic studies of humans (Cotton et al., “Validation of the AMPFISTR (R) SGM Plus (TM) System for Use in Forensic Casework,” Forensic Sci. Int'l, 112:151-161 (2000)) and has further proven effective for determining sex in several hominoid primates, including gorillas, chimpanzees, and gibbons (Bradley et al., “Accurate DNA-based Sex Identification of Apes Using Non-invasive Samples,” Cons. Genetics, 2:179-181 (2001); Ensminger & Hoffman, “Sex Identification Assay Useful in Great Apes is Not Diagnostic in a Range of Other Primate Species,” Am. J. Primatol., 56:129-134 (2002)), the procedure, unfortunately, is not broadly applicable across the rest of the primate order. For example, the assay is ineffective for assigning sex in black lemurs (Eulemur macaco), baboons (Papio), mandrills (Mandrillus), and a host of New World monkeys, including spider monkeys (Ateles), woolly monkeys (Lagothrix), squirrel monkeys (Saimiri), and tamarins (Saguinus), and it is likewise ineffective for the remaining hominoid, the orangutan (Pongo) (Bradley et al., “Accurate DNA-based Sex Identification of Apes Using Non-invasive Samples,” Cons. Genetics, 2:179-181 (2001); Ensminger & Hoffman, “Sex Identification Assay Useful in Great Apes is not Diagnostic in a Range of Other Primate Species,” Am. J. Primatol., 56:129-134 (2002); Steiper & Ruvolo, “Genetic Sex Identification of Orangutans,” Anthropologischer Anzeiger, 61:1-5 (2003)).

Additionally, while molecular techniques are increasingly being employed in primate field studies (see reviews in Di Fiore, “Molecular Genetic Approaches to the Study of Primate Behavior, Social Organization, and Reproduction,” Yrbk. Phys. Anthropol., 46:62-99 (2003) and De Ruiter, “Genetic Markers in Primate Studies: Elucidating Behavior and Its Evolution,” Int. J. Primatol., 25:1173-1189 (2004)), as yet, few methods have been forwarded for reliably typing the sex of nonhuman primate DNA, particularly that recovered from samples such as feces which is likely to be degraded or highly fragmented. For primate conservation geneticists and molecular ecologists, the ability to determine the sex of noninvasively collected samples would clearly be beneficial for many kinds of studies, such as tracking demographic changes in population sex ratios over time, determining the sex composition of unhabituated social groups, examining sex variation in fecal pathogen loads, etc.

The present invention is directed to overcoming these and other deficiencies in the art.

### SUMMARY OF THE INVENTION

One aspect of the present invention is a method for identifying the sex of a primate. The method involves providing a biological sample collected from the primate. The biological sample is contacted with one or more probes that hybridize to a target SRY nucleic acid molecule at locations within a region spanning nucleotide 349 and nucleotide 513 of the nucleotide sequence of SEQ ID NO: 40 and/or its complement, under conditions effective to permit hybridization of the probes to any of the locations, if present, in the sample. Any hybridization of the one or more probes to any of the locations is detected and the sex of the primate is identified based on whether or not any hybridization occurs.

Another aspect of the present invention relates to an oligonucleotide probe that hybridizes to a target SRY nucleic acid molecule at locations within a region spanning nucleotide 349 and nucleotide 513 of the nucleotide sequence of SEQ ID NO: 40 and /or its complement.

Yet another aspect of the present invention is drawn to an oligonucleotide probe that hybridizes to a target nucleic acid molecule at locations within a region spanning nucleotide 155 and nucleotide 323 of the nucleotide sequence of SEQ ID NO: 41 and /or its complement.

Still another aspect of the present invention relates to an oligonucleotide probe that comprises the nucleotide sequence of SEQ ID NO: 9.

Still another aspect of the present invention relates to an oligonucleotide probe that hybridizes to a target nucleic acid molecule at locations within a region spanning nucleotide 158 and nucleotide 350 of the nucleotide sequence of SEQ ID NO: 42 and /or its complement.

Yet another aspect of the present invention relates to a method for identifying the sex of a primate. This method involves providing a biological sample collected from the primate. The biological sample is contacted with two or more different probes that hybridize to locations within a region of a target amelogenin nucleic acid molecule of an X chromosome and that hybridize to locations within a region of a target amelogenin nucleic acid molecule of a Y chromosome under conditions effective to permit hybridization between the two or more probes and the locations, if present, in the sample. In this method, the region of a target amelogenin nucleic acid molecule of the X chromosome and the region of a target amelogenin nucleic acid molecule of the Y chromosome have different lengths, and the region of a target amelogenin nucleic acid molecule of the X chromosome spans nucleotide 125 and nucleotide 323 of the nucleotide sequence of SEQ ID NO: 41 and/or its complement. Any hybridization of the two or more different probes to any of the locations is detected using polymerase chain reaction conditions and the sex of the primate is identified.

Molecular ecological studies can provide insights into the mating system, dispersal pattern, social organization, and population structure of wild primates, all of which can be important for guiding conservation policy. For such studies, it would be useful if researchers were able to reliably identify the sex of noninvasively sampled animals, but while several genetic methods for identifying the sex of primate DNA samples have been developed for humans, few of these are applicable across the primate order. Presented here is a simple method for sex typing primate DNA that can involve a multiplex polymerase chain reaction, which, at the same time, amplifies small portions of the X-linked enamel protein gene amelogenin and the Y-linked sex-determining region gene SRY. The larger X chromosome amplicon serves as a positive PCR control, while the Y chromosome amplicon determines whether the sample is male or female. The procedure has been tested and shown to be effective for sexing a wide breadth of primate taxa and for reliably sexing DNA extracted from noninvasively collected hair and fecal samples.

Significantly, and advantageously, the methods and probes of the present invention are effective for sex-typing a wide breadth of primate taxa and for reliably sex-typing DNA extracted from noninvasively collected samples.

### BRIEF DESCRIPTION OF THE DRAWINGS

FIGS. 1A-E show the alignment of DNA sequences for the X chromosome copy of the amelogenin gene of humans (Homo) (i.e., Homo(X1 4440) (SEQ ID NO: 1), HomoAMGX(M55418) (SEQ ID NO: 2)), chimpanzees (Pan) (i.e., PanAMGX (SEQ ID NO: 3)), squirrel monkeys (Saimiri) (i.e., SaimiriAMGX(U88981) (SEQ ID NO: 4)), baboons (Papio) (i.e., PapioAMGX (SEQ ID NO: 5)), and orangutans (Pongo) (i.e., PongoAMGX (SEQ ID NO: 6)) (all sequences published on and downloaded from GenBank). Also shown is the alignment of Sullivan et al.'s (Sullivan et al., “A Rapid and Quantitative DNA Sex Test: Fluorescence-based PCR Analysis of X-Y Homologous Gene Amelogenin,” BioTechniques, 15:637-641 (1993), which is hereby incorporated by reference in its entirety) amelogenin-A primer (i.e., AMELA→(SEQ ID NO: 7)) and the complement (SEQ ID NO: 8) of the amelogenin-B primer (i.e., AMELB←), and the anthropoid amelogenin forward primer (i.e., DiFioreAMEL-F1→(SEQ ID NO: 9)) and the complement (SEQ ID NO: 10, Where N is A or T) of the anthropoid amelogenin reverse primer (i.e., DiFioreAMEL-R1→(GCTGGGNTAGAACCAAGCTG (SEQ ID NO: 11, where N is A or T))) of the present invention. Single black dots represent mismatches across taxon and/or primer sequences.

FIGS. 2A-B are schematic overviews of aligned primate sequence data for a portion of the amelogenin X (FIG. 2A) and SRY (FIG. 2B) loci, indicating the locations of the PCR primers used herein and the corresponding expected amplicons. These alignments incorporate at least one exemplar of every primate genus for which appropriate amelogenin X or SRY sequence data are currently available on GenBank (accession numbers listed), which were used to the design the PCR primers disclosed herein.

FIGS. 3A-N show the alignment of DNA sequences for the Y chromosome SRY gene of common chimpanzees (Pan troglodytes) (i.e., PantroglodytesSRY(AF008917) (SEQ ID NO: 12), PantroglodytesSRY(AJ222687) (SEQ ID NO: 13), PantroglodytesSRY(X86380) (SEQ ID NO: 14)), gorillas (Gorilla) (i.e., GorillaSRY(AJ003068) (SEQ ID NO: 15), GorillaSRY(X86382) (SEQ ID NO: 16)), Asian colobine monkeys (Presbytis, trachypithecus) (i.e., PresbytisSRY (SEQ ID NO: 17), TrachypithecusSRY (SEQ ID NO: 18)), humans (Homo) (i.e., HomoSRY(L08063) (SEQ ID NO: 19), HomoSRY(L10102) (SEQ ID NO: 20), HomoSRY(X53772) (SEQ ID NO: 21), HomoSRY(L10101) (SEQ ID NO: 22)), capuchin monkeys (Cebus) (i.e., CebusSRY (SEQ ID NO: 23), bonobos (Pan paniscus) (i.e., Pan paniscus SRY (X86381) (SEQ ID NO: 24)), orangutans (Pongo) (i.e., PongoSRY(X86383) (SEQ ID NO: 25)), marmosets (Callithrix) (i.e., CallithrixSRY(X86386) (SEQ ID NO: 26)), baboons (Papio) (i.e., Papio hamadrayas sry (X86385) (SEQ ID NO: 27)), gibbons (Hylobates) (i.e., HylobatesSRY(X86384) (SEQ ID NO: 28)), and macaques (Macaca) (i.e., MacacaSRY(Z26906) (SEQ ID NO: 29)) (all sequences published on and downloaded from GenBank). Also shown is the alignment of Santos et al.'s (Santos et al., “Reliability of DNA-Based Sex Tests,” Nature Genetics, 18:103 (1998), which is hereby incorporated by reference in its entirety) F11 primer (i.e., F11→(SEQ ID NO: 30)) and the complement (SEQ ID NO: 31) of the R7 primer (i.e., R7→), and the SRY forward primer (i.e., Di Fiore SRY-F1→(SEQ ID NO: 32)) and the complement (SEQ ID NO: 33) of the SRY reverse primer (i.e., Di Fiore SRY-R1→(TGTGCCTCCTGGAAGAATGG (SEQ ID NO: 34)) of the present invention. Single black dots represent mismatches across taxon and/or primer sequences.

FIGS. 4A-4D show amelogenin and SRY banding patterns for male and female primates. FIG. 4A depicts idealized electropherograms showing the expected banding pattern for males versus females in catarrhine and platyrrhine primates based on published amelogenin and SRY sequence data available on GenBank for Homo, Pan, Pongo, Papio, and Saimiri. FIGS. 4B-4D show the results of the sexing assay of the present invention applied to high quality DNA from hominoids (apes) (FIG. 4B), cercopithecoids (Old World monkeys) (FIG. 4C), and platyrrhines (New World monkeys) (FIG. 4D). Only a representative subset of assayed taxa and samples are shown. The hominoid lanes (FIG. 4B, left to right) are: Homo sapiens male, Homo sapiens female, Pan troglodytes unknown (assigned male), Pongo pygmaeus unknown (assigned male), Pongo pygmaeus unknown (assigned female), Pongo pygmaeus unknown (assigned female), Gorilla gorilla unknown (assigned female), Gorilla gorilla unknown (assigned female), Hylobates agilis male, and Symphalangus syndactylus male. The cercopithecoid lanes (FIG. 4C, left to right) are: Homo sapiens male (control), Macaca sp. male, Chlorocebus aethiops male, Chlorecebus aethiops female, Cercocebus torquatus male, Cercopithecus ascanius male, Cercopithecus sp. female, Papio sp. male, Papio sp. female, Mandrillus sphinx male, Mandrillus sphinx female, Theropithecus gelada male, Colobus guereza unknown (assigned female), Procolobus badius male, Nasalis larvatus unknown (assigned female), Pygathrix nemaeus male, and Presbytis melalophos male. The platyrrhine lanes (FIG. 4D, left to right) are: Homo sapiens male (control), Ateles belzebuth male, Ateles belzebuth female, Lagothrix lagotricha male, Lagothrix lagotricha female, Alouatta seniculus male, Alouatta seniculus female, Pithecia pithecia male, Chiropotes satanus female, Callicebus donacophilus male, Callithrix pygmaea male, Saguinus oedipus male, Cebus albifrons unknown (assigned male), Saimiri sciureus male, Saimiri sciureus unknown (assigned female), and Aotus lemurinus female.

FIG. 5 shows exemplary results of a sexing assay in accordance with the present invention applied to DNA extracted from fecal samples of two platyrrhine genera. Samples, from left to right, include five known Ateles belzebuth individuals (Oko (male), Kuraka (female), Oso (male), Toma (female), and Kaya (female)) and one Lagothrix lagotricha male.

FIGS. 6A-D show the alignment of DNA sequences for the X chromosome copy of the amelogenin gene of two strepsirhine primates: Lemur (i.e., LemurAMGX (SEQ ID NO: 35)) and Otolemur (i.e., OtolemurAMGX (SEQ ID NO: 36)) (all sequences published on and downloaded from GenBank). Also shown is the alignment of the anthropoid amelogenin forward primer (i.e., DiFioreAMEL-F1→(SEQ ID NO: 9)) and the complement (SEQ ID NO: 10, where N is A or T) of the anthropoid amelogenin reverse primer (i.e., DiFioreAMEL-R1←(GCTGGGNTAGAACCAAGCTG (SEQ ID NO: 11, where N is A or T))) of the present invention, and the strepsirhine amelogenin forward primer (i.e., DiFioreAMEL-F1 (strep) (SEQ ID NO: 37)) and the complement (SEQ ID NO: 38, where N is C or T) of the strepsirhine amelogenin reverse primer (i.e., DiFioreAMEL-RI (strep) (AACATCNTACCTAATCCCCACA (SEQ ID NO: 39, where N is C or T))) of the present invention. Single black dots represent mismatches across taxon and/or primer sequences.

FIGS. 7A-7B show amelogenin and SRY banding patterns for male and female strepsirhine primates. FIG. 7A shows an idealized electropherogram for strepsirhine primates based on published sequence data in GenBank for Lemur and Otolemur. FIG. 7B shows the results of a sexing assay of the present invention applied to high quality DNA from tarsiers and four strepsirhine genera. Taxa shown are, from left to right, Tarsius syrichta female, Otolemur crassicaudatus female, Mirza coquereli unknown (assigned male), Lemur catta male, and Daubentonia madagascariensis male.

FIG. 8 is an array view (bottom right) and electropherograms (top and bottom left) showing the results of the sexing assay with fluorescently-labeled primers described in Example 6 applied to 22 high quality DNA samples from 18 catarrhine genera. The sample number, genus, and reported sex (M=male, F=female, U=unknown) of each sample are noted at the top of each electropherogram panel, and the assigned sex is indicated by the symbol at the bottom right.

FIG. 9 shows exemplary results of a sexing assay in accordance with the present invention using fluorescently-labeled primers applied to DNA extracted from blood and feces of two platyrrhine genera and visualized on an ABI 3730 automated DNA analysis system. Samples, from top to bottom in the array view, include a negative control, two female spider monkeys (Ateles), one male spider monkey (Ateles), and one male howler monkey (Alouatta). Electropherograms of the DNA samples themselves appear to the right. In both the array view and the electropherograms, a DNA size standard appears in red, while the X and Y chromosome fragments amplified in the assay appear in blue and green, respectively.

### DETAILED DESCRIPTION OF THE INVENTION

The present invention relates to a method for identifying the sex of a primate. The method involves providing a biological sample collected from the primate. The biological sample is contacted with one or more probes that hybridize to locations within a target SRY nucleic acid molecule at a region spanning nucleotide 349 and nucleotide 513 of the nucleotide sequence of SEQ ID NO: 40 and/or its complement, under conditions effective to permit hybridization of the probes to any of the locations, if present, in the sample. Any hybridization of the one or more probes to any of the locations is detected and the sex of the primate is identified based on whether or not any hybridization occurs.

The primate SRY consensus sequence has a nucleotide sequence of SEQ ID NO: 40, as follows:

nggnggtnnncnggttgggnggngttgannggggtgntgngggcggag 48
aaangnaagttncattacnaaagttaangtaacaangaanntggtnga 96
agtnantttnggatagtnaantnagtttcnnantctgncanctttnan 144
ntttnnngnannctncttgtttttgacaatgcantcntangcttcnnc 192
natgntnagngtattnaacnnngatnnntacantccagntgngcnana 240
gantnnnccngntnnnngganaagctcttccntcntttgnactganan 288
ctntanctcnaagnatcngngnnaancnggagaaaacagtaaagnnan 336
cgtccagnanngagtgaagcgacccatgaacgcnttcntngtgtggtc 384
tcgngancanaggcgcaagatggntntagagaatcccnnnatgcgaaa 432
ntcnganatcagcaagcngctgggataccngtggaaanngcttacngn 480
agccganaaatggccattcttccaggaggcacagaaantncaggccat 528
gcanagagagaaatacccnaattataagtatcgacctcgtcggaaggc 576
naanntnctgcnnaanantnncagttngcntncngnngatncnncttc 624
ngnncnctgnnnnnaantgnnnnannnnnannacngnttgtnca, 668

where n at positions 1, 8, 12, 52, 84, 114, 117, 181, 212, 220, 237, 298, 370, 374, 470, 487, 581, 627, and 666 is A or T; n at positions 4, 9, 23, 40, 67, 94, 100, 102, 119, 144, 178, 191, 199, 219, 225, 245, 261, 288, 294, 314, 316, 336, 344, 394, 423, 424, 436, 439, 450, 462, 520, 547, 583, 589, 594, 597, 625, 628, 635, 636, 638, 641, 656, and 661 is A or G; n at positions 20, 127, 129, 151, 202, 243, 252, 306, 347, 408, 422, 480, 588, 608, 613, and 659 is A or C; n at positions 134 and 333 is A, T, or G; n at positions 54, 255, 478, and 637 is A, T, or C; n at positions 142, 275, 286, 291, and 655 is A, G, or C; n at positions 30 and 649 is A or absent; n at positions 646 and 652 is A, G, or absent; n at positions 60, 106, 149, 218, 272, 302, 596, 606, and 620 is T or G; n at positions 37, 88, 89, 137, 145, 150, 153, 155, 156, 184, 190, 193, 197, 208, 214, 234, 246, 247, 250, 254, 256, 308, 334, 376, 388, 391, 433, 471, 518, 532, 603, 612, 617, 630, and 634 is T or C; n at positions 310, 346, and 580 is T, G, or C; n at position 645 is T or absent; n at positions 644 and 651 is T, C, or absent; n at positions 10, 29, 76, 126, 159, 213, 231, 239, 257, 280, 311, 410, 577, and 592 is G or C; n at position 653 is G or absent; n at positions 647 and 650 is G, C, or absent; and n at positions 610 and 619 is any nucleotide.

In this aspect of the present invention, lack of hybridization between the probes and the target SRY nucleic acid molecule identifies the primate as a female. Hybridization between the probes and the target SRY nucleic acid molecule identifies the primate as a male.

Suitable probes according to all aspects of the present invention include, for example, oligonucleotide sequences, complementary DNA and RNA, and peptide nucleic acids.

In accordance with the methods of the present invention, the target SRY nucleic acid molecule is, preferably, 150-300 nucleotides in length, although target nucleic acid molecules smaller than 150 nucleotides and target nucleic acid molecules larger than 300 nucleotides are also contemplated by the present invention. The locations of the target SRY nucleic acid molecule to which the probes hybridize are, preferably, at least 20 nucleotides in length, although slightly smaller and slightly larger locations are also contemplated.

Suitable probes according to this and all aspects of the present invention include, for example, probes that comprise at least 45% G+C bases, have an estimated melting temperature of at least 58° C., and/or display minimal 3′ self-complementarity and ability to form dimers with other probes.

In aspects of the present invention in which the biological sample is contacted with more than one probe, the probes may be designed such that each probe has a greater hybridizing affinity for the target SRY nucleic acid molecule than their hybridizing affinity for other probes contacted with the sample.

Suitable probes that hybridize to a target SRY nucleic acid molecule according to this and all aspects of the present invention include, for example, 5′-AGT GAA GCG ACC CAT GAA CG-3′ (SEQ ID NO: 32) and 5′-TGT GCC TCC TGG AAG AAT GG-3′ (SEQ ID NO: 34).

Suitable primates include those with known or published primate SRY sequences, including, for example, Strepsirhini, Lemuroidea, Lemur, Lemur catta, Mirza coquereli, Daubentonia madagascariensis, Lorisoidea, Otolemur, Otolemur crassicaudatus, Otolemur garnetti, Haplorhini, Hominoidea, Homo, Homo sapiens, Pan, Pan troglodytes, Pongo, Pongo pygmaeus, Gorilla gorilla, Hylobates agilis, Symphalangus syndactylus, Cercopithecoidea, Cercopithecinae, Macaca sp., Cercopithecus sp., Cercopithecus ascanius, Cercopithecus nictitans, Cercocebus torquatus, Chlorocebus aethiops, Lophocebus aterrimus, Allenopithecus nigroviridis, Erythrocebuspatas, Papio sp., Theropithecus gelada, Mandrillus leucophaeus, Mandrillus sphinx, Colobinae, Semnopithecus, Procolobus badius, Colobus guereza, Presbytis melalophos, Nasalis larvatus, Pygathrix nemaeus, Platyrrhini, Cebidae, Cebus albifrons, Saimiri, Saimiri sciureus, Aotus vociferans, Aotus lemurinus, Saguinus oedipus, Leontopithecus, Leontopithecus rosalia, Cebuella pygmaea, Pithecidae, Pithecia pithecia, Chiropotes satanus, Callicebus discolor, Callicebus donacophilus, Atelidae, Lagothrix, Lagothrix lagotricha, Ateles, Ateles belzebuth, Alouatta seniculus, Tarsioidea, Tarsiidae, and Tarsius syrichta.

Suitable biological samples according to the present invention include, for example, hair, feces, blood, tissue, urine, saliva, cheek cells, skin, for example skin cells contained in fingerprints, and semen. It is contemplated that samples may be collected invasively or noninvasively.

Hybridization may be carried out according to methods described in the art, for example, those described in Sambrook et al., Molecular Cloning: A Laboratory Manual, Third Edition, Cold Spring Harbor: Cold Spring Harbor Laboratory Press, New York (2001), which is hereby incorporated by reference in its entirety. Generally, a sample is subjected to conditions under which DNA present in the sample is denatured and/or digested. The DNA is mixed with hybridization probes under conditions in which the hybridization probes anneal with any complementary single-stranded DNA present in the sample. The precise conditions for any particular hybridization are left to those skilled in the art, because there are variables involved in nucleic acid hybridizations beyond those of the specific nucleic acid molecules to be hybridized that affect the choice of hybridization conditions. These variables include: the substrate used for nucleic acid hybridization (e.g., charged vs. non-charged membrane); the detection method used (e.g., radioactive vs. chemiluminescent); the source and concentration of the nucleic acid involved in the hybridization, the annealing temperature used, the precise buffer conditions used, the concentration of reactants in the reaction mix used, the ratio of probes to sample DNA, and the use of additives specific for the hybridization method being used. All of these variables are routinely taken into account by those skilled in the art prior to undertaking a nucleic acid hybridization procedure.

Contacting may be carried out by methods apparent to one of ordinary skill in the art. This may involve an amplification procedure in which the target nucleic acid molecule is amplified prior to detecting. Suitable methods for amplification of the target nucleic acid molecule include, but are not limited to, polymerase chain reaction (PCR), ligase chain reaction (LCR) (Barany, Proc. Nat'l Acad. Sci. U.S.A., 88, 189 (1991), which is hereby incorporated by reference in its entirety), ligase detection reaction (LDR), LDR-PCR, strand displacement amplification (Walker et al., Nucleic Acids Res, 20, 1691 (1992); Walker et al., Proc. Nat'l Acad. Sci. U.S.A., 89, 392 (1992), which are hereby incorporated by reference in their entirety), transcription-based amplification (Kwoh et al., Proc. Nat'l Acad. Sci. US.A., 86, 1173 (1989), which is hereby incorporated by reference in its entirety), self-sustained sequence replication (or “3SR”) (Guatelli et al., Proc. Nat'l Acad. Sci. U.S.A., 87, 1874 (1990), which is hereby incorporated by reference in its entirety), nucleic acid transcription-based amplification system (TAS), the Q-beta replicase system (Lizardi et al., Biotechnology, 6, 1197 (1988), which is hereby incorporated by reference in its entirety), hybridization signal amplification (HSAM), nucleic acid sequence-based amplification (NASBA) (Lewis, R., Genetic Engineering News, 12(9), 1 (1992), which is hereby incorporated by reference in its entirety), the repair chain reaction (RCR) (Lewis, R., Genetic Engineering News, 12(9), 1 (1992), which is hereby incorporated by reference), boomerang DNA amplification (BDA) (Lewis, R., Genetic Engineering News, 12(9), 1 (1992), which is hereby incorporated by reference in its entirety), and branched-DNA methods.

Preferably amplification is carried out under PCR, more preferably, multiplex PCR, conditions. PCR generally involves the use of a pair of probes, or more than one pair of probes (multiplex PCR) which specifically bind to the nucleic acid molecule(s) of interest, but do not bind to other nucleic acid molecules, under the same hybridization conditions, and which serve as primers for the amplification reaction. In at least one aspect of the present invention, a biological sample is contacted with one or more probes that hybridize to a target SRY nucleic acid molecule and contacted with one or more probes that hybridize to a target amelogenin nucleic acid molecule. Either or both contacting steps may be carried out under polymerase chain reaction conditions and may be performed simultaneously. In at least one embodiment of the present invention, the region of the SRY nucleic acid molecule subjected to PCR conditions, if present in the sample, is smaller than the region of the amelogenin nucleic acid molecule subjected to PCR conditions, if present in the sample.

Detecting may be carried out by, for example, Northern blot (Thomas, P. S., “Hybridization of Denatured RNA and Small DNA Fragments Transferred to Nitrocellulose,” Proc. Nat'l. Acad. Sci. USA, 77:5201-05 (1980), which is hereby incorporated by reference in its entirety), Southern blot (Southern, “Detection of Specific Sequences Among DNA Fragments Separated by Gel Electrophoresis,” J. Mol. Biol., 98:503-17 (1975), which is incorporated herein by reference in its entirety), PCR, multiplex PCR (Erlich, et. al., “Recent Advances in the Polymerase Chain Reaction”, Science 252:1643-51 (1991), which is incorporated herein by reference in its entirety), in-situ hybridization (Nucleic Acid Hybridization: A Practical Approach, Haimes and Higgins, Eds., Oxford:IRL Press (1988), which is hereby incorporated by reference in its entirety), in-situ PCR (Haase et al., “Amplification and Detection of Lentiviral DNA Inside Cells,” Proc. Natl. Acad. Sci. USA, 87(13):4971-5 (1991), which is hereby incorporated by reference in its entirety), or other suitable hybridization assays known in the art. In aspects of the present invention in which contacting involves amplification of the target nucleic acid, detecting may be carried out using any method commonly associated with the method of amplification selected by the user, including, but not limited to, gel electrophoresis, array-capture, and direct sequencing.

The probes in this and all aspects of the present invention can be labeled or tagged in accordance with the detection method of choice. Nucleic acid probes are generally “tagged” using traditional radioactive labeling and detection methods (Sambrook et al., Molecular Cloning: A Laboratory Manual, Cold Spring Laboratory, Cold Spring Harbor, New York (1989), which is hereby incorporated by reference in its entirety), or with non-radioactive materials, such as biotin, digoxigenin, various fluorochromes, or haptens (Hybridization with cDNA Probes User Manual, Clonetech Laboratories, Calif. (2000); Harvey, et al., Protocols for Nucleic Acid Analysis by Nonradioactive Probes, Ed. P. G. Isaac, Humana Press, New Jersey, pp. 93-100 (1994), which are hereby incorporated by reference in their entirety). Suitable labels also include, without limitation, fluorescent labels, radioactive labels, nuclear magnetic resonance active labels, bioluminescent labels, and chromophore labels. The labeling method and assay conditions will be dictated by the choice of assay system to be employed.

The method of the present invention may further involve contacting the biological sample with one or more probes that hybridize to a target amelogenin nucleic acid molecule at locations within a region spanning nucleotide 125 and nucleotide 323 of the nucleic acid of an anthropoid amelogenin consensus sequence (i.e. SEQ ID NO: 41) and/or its complement, and/or at locations within a region spanning nucleotide 158 and nucleotide 350 of the nucleic acid of the strepsirhine amelogenin consensus sequence (i.e. SEQ ID NO: 42) and/or its complement. The biological sample is contacted with the probes under conditions effective to permit hybridization of the probes to any of the locations, if present, in the sample. Any hybridization of the one or more probes that hybridize to a target amelogenin nucleic acid molecule is detected.

The anthropoid amelogenin consensus sequence has a nucleotide sequence of SEQ ID NO: 41, as follows:

gtaatttttctctttactaattttgaccattgtttgcgttaacantgc 48
cctgggctctgtaaagaatagtgtgttgattcttnatcccagatgttt 96
ctcaagnggncctgattttacagttnctaccaccagcttcccngttta 144
agctctnganggttggcctcaagcctgtgtngtcccagcancctccng 192
cctggcnactctgactcagtctntcctcctaaatatgncngtnanctt 240
acccatcatgaaccacnnntnagggaggctcnatgntagggcaaaaag 288
tnaactctgacnnnncagcttggttctancccagctantaaaangtaa 336
ggattaggtaagatgttatttaanantntttccagctcaanaaactnc 384
tgattctaagatagtcacactntanntgtgtctctnnnttgnctctgc 432
tgaaatattantgactaagtggtatangagagantcngcanaacanng 480
naatgcatgangttttggncntnnggtttgaggttctcctcaanctcn 528
tactaactntntgantttgggcaantcatttnctntttctggaaccct 576
ggtttcctcatntggagaaaggaaatnattataatnannatatntcaa 624
aatattgtttggagantaatatanttaannnatatgaaaagtnctttg 672
tcaantataatatgagcaannttact, 698

where n at positions 139, 317, 553, and 620 is A or T; n at positions 45, 230, 235, 258, 276, 326, 360, 410, 421, 478, 481, 499, 501, 504, 539, 603, 640, 654, 655, 667, and 692 is A or G; n at positions 290 and 364 is A or C; n at position 648 is A, G, or C; n at positions 302 and 612 is A or absent; n at position 377 is A, C, or absent; n at positions 443, 459, 473, 537, and 693 is T or G; n at positions 83, 103, 106, 122, 154, 175, 191, 199, 232, 257, 272, 332, 362, 383, 409, 420, 422, 466, 469, 479, 503, 524, 528, 543, 563, and 653 is T or C; n at positions 151 and 300 is T or absent; n at positions 185, 215, 237, 259, 261, 406, 426, 588, and 677 is G or C; n at position 301 is G or absent; and n at positions 303, 491, 560, 614, and 615 is C or absent.

The strepsirhine amelogenin consensus sequence has a nucleotide sequence of SEQ ID NO: 42, as follows:

gtaatttttctntttactaantttgaccattntttgnnttancaatgc 48
cntggngctctgtaaagaatagtgtgttgattcttcatncannntntt 96
tctnnaataatcccaattttacagntcntaccaccagnttncnagttt 144
aanccctganggntggcctcaagcctgcattgnnccagcancctncta 192
cctggccactctnagnctntcctcctnaanannnnnataatnttatct 240
ntnatgaacnaccacttagggaggctnncannntagggnngaaagaga 288
antctggctgaananccttgntntgtcccagccagtanaatgtgggga 336
ttaggtangatgttatntaagnttttttccnagctcnanaaactcctg 384
attntaagacattnacacttgatgtnngtcnctcacntgncttcactg 432
aaagatgagtgnctnagtgcnnnatgagggnctntgcagaggnatgga 480
aancancaganntcnnngtntcnggtttgaggtnctnctcaatccctt 528
nctaactncangacntngggaaaancattttctctctctggaactttg 576
gntccctcanntggagaagnnaaatanntatgatnannanngcatatt 624
tcaaaanattgnttggaaagtaanananttattgantatgaaaagtgt 672
ttnntcaagtataacttgagnaangttaannannanntnntanat, 717

where n at positions 154, 177, 250, 358, 411, 421, 475, 603, and 648 is A or T; n at positions 32, 37, 42, 54, 90, 92, 94, 101, 147, 157, 241, 272, 280, 303, 344, 375, 424, 444, 454, 463, 483, 491, 497, 503, 529, 536, 596, 597, 652, 676, and 696 is A or G; n at positions 227, 388, 447, 500, 660, 693, and 703 is A or C; n at positions 219, 367, 373, 611, and 617 is A or absent; n at positions 91, 234, 539, 586, and 604 is T or G; n at positions 12, 21, 50, 87, 100, 124, 134, 137, 139, 178, 185, 189, 208, 243, 271, 279, 290, 301, 309, 353, 398, 415, 453, 455, 486, 492, 495, 514, 517, 543, 545, 553, 578, 587, 631, 636, 650, 675, and 715 is T or C; n at positions 121, 224, 228, 616, 702, 705, 706, 708, 709, and 712 is T or absent; n at positions 38, 205, 211, 268, 273, 311, 326, 466, and 496 is G or C; n at positions 225, 226, 410, 613, and 711 is G or absent; and n at positions 222, 267, and 614 is C or absent.

In this aspect of the present invention, lack of hybridization between the probes that hybridize to a target SRY nucleic acid molecule and the target SRY nucleic acid molecule with hybridization between the probes that hybridize to a target amelogenin nucleic acid molecule and the target amelogenin nucleic acid molecule identifies the primate as a female. Hybridization between the probes that hybridize to a target SRY nucleic acid molecule and the target SRY nucleic acid molecule with hybridization between the probes that hybridize to a target amelogenin nucleic acid molecule and the target amelogenin nucleic acid molecule identifies the primate as a male.

Suitable probes that hybridize to a target amelogenin nucleic acid molecule include, for example, 5′-ACC ACC AGC TTC CCA GTT TA-3′ (SEQ ID NO: 9), 5′-GCT GGG NTA GAA CCA AGC TG-3′ (SEQ ID NO: 11, where N is any A or T), 5′-TGG CCT CAA GCC TGC ATT-3′(SEQ ID NO: 37), 5′-AAC ATC NTA CCT AAT CCC CAC A-3′ (SEQ ID NO: 39, where N is C or T), and combinations thereof.

In at least one preferred embodiment of this aspect of the present invention, contacting is carried out by a single, multiplex PCR to amplify a short fragment of the amelogenin gene from the X chromosome (plus, potentially, its homologue on the Y) and, at the same time, a portion of the single-copy sex-determining region (SRY) gene on the Y chromosome, with the probes of the present invention acting as primers in the PCR reaction. The amelogenin locus is expected to amplify in all samples containing sufficient nuclear DNA (since all individuals, male or female, will possess the X template), while the SRY locus should amplify only if a Y chromosome template is present. Thus, the amelogenin X fragment serves as a positive PCR control, while the SRY fragment is used to assign sex. The X and Y fragments amplified differ in size by ˜35 base pairs and are easily separated and visualized using benchtop gel electrophoresis and post-staining. The method thus requires no specialized laboratory equipment such as automated DNA sequencers, although the technique could easily be modified to take advantage of those facilities by using primers bearing any of various labels (e.g., radioactive, fluorescent, or bioluminescent labels) as dictated by the choice of assay system to be employed. Importantly, the target fragments are short (˜200 base pairs or less) and should amplify reliably even from the degraded DNA templates typically recovered from noninvasively collected samples.

The present invention also relates to a method for identifying the sex of a primate. This method involves providing a biological sample collected from the primate. The biological sample is contacted with two or more different probes that hybridize to locations within a region of a target amelogenin nucleic acid molecule of an X chromosome and that hybridize to locations within a region of a target amelogenin nucleic acid molecule of a Y chromosome under conditions effective to permit hybridization between the two or more probes and the locations, if present, in the sample. In this method, the region of a target amelogenin nucleic acid molecule of the X chromosome and the region of a target amelogenin nucleic acid molecule of the Y chromosome have different lengths, and the region of a target amelogenin nucleic acid molecule of the X chromosome spans nucleotide 125 and nucleotide 323 of the nucleotide sequence of SEQ ID NO: 41 and/or its complement. Any hybridization of the two or more different probes to any of the locations is detected using polymerase chain reaction conditions and the sex of the primate is identified.

Hybridization and polymerase chain reaction may be carried out as described above.

Suitable probes according to this aspect of the present invention include, for example, SEQ ID NO: 9 and SEQ ID NO: 11. This amelogenin primer pair is likely to simultaneously amplify the homologous Y chromosome copy of that gene in most anthropoid primates, and in some taxa the Y homolog differs slightly in size from the X fragment. Therefore, when amplified by, for example, polymerase chain reaction, a product of one length identifies the primate as a female, and two products of different lengths identifies the primate as a male.

The present invention also relates to oligonucleotide probes that hybridize to a target nucleic acid molecule at locations within a region spanning nucleotide 349 and nucleotide 513 of SEQ ID NO: 40 and /or its complement.

Suitable probes according to this aspect of the present invention include, for example, SEQ ID NO: 32 and SEQ ID NO: 34.

The present invention also relates to oligonucleotide probes that hybridize to a target nucleic acid molecule at locations within a region spanning nucleotide 155 and nucleotide 323 of SEQ ID NO: 41 and /or its complement.

The locations of the target amelogenin nucleic acid molecule to which the probes hybridize are, preferably, at least 20 nucleotides in length, although smaller locations are also contemplated by the present invention.

Suitable probes according to this and all aspects of the present invention include, for example, probes that comprise at least 45% G+C bases, have an estimated melting temperature of at least 58° C., and/or display minimal 3′ self-complementarity and ability to form dimers with other probes.

Suitable probes according to this aspect of the present invention include, for example, SEQ ID NO: 11.

Still another aspect of the present invention relates to an oligonucleotide probe that comprises the nucleotide sequence of SEQ ID NO: 9.

The present invention also relates to oligonucleotide probes that hybridize to a target nucleic acid molecule at locations within a region spanning nucleotide 158 and nucleotide 350 of SEQ ID NO: 42 and/or its complement.

The locations of the target amelogenin nucleic acid molecule to which the probes hybridize are, preferably, at least 20 nucleotides in length, although smaller locations are also contemplated by the present invention.

Suitable probes according to this and all aspects of the present invention include, for example, probes that comprise at least 45% G+C bases, have an estimated melting temperature of at least 58° C., and/or display minimal 3′ self-complementarity and ability to form dimers with other probes.

Suitable probes according to this aspect of the present invention include, for example, SEQ ID NO: 37 and SEQ ID NO: 39.

The present invention may be further illustrated by reference to the following examples.

### EXAMPLES

### Example 1

### Design Of Anthropoid Primers

Based on published sequence data for primates, PCR primers were designed to amplify small portions of the amelogenin X and SRY genes. The goal was to create a set of universal primers, applicable across most primates.

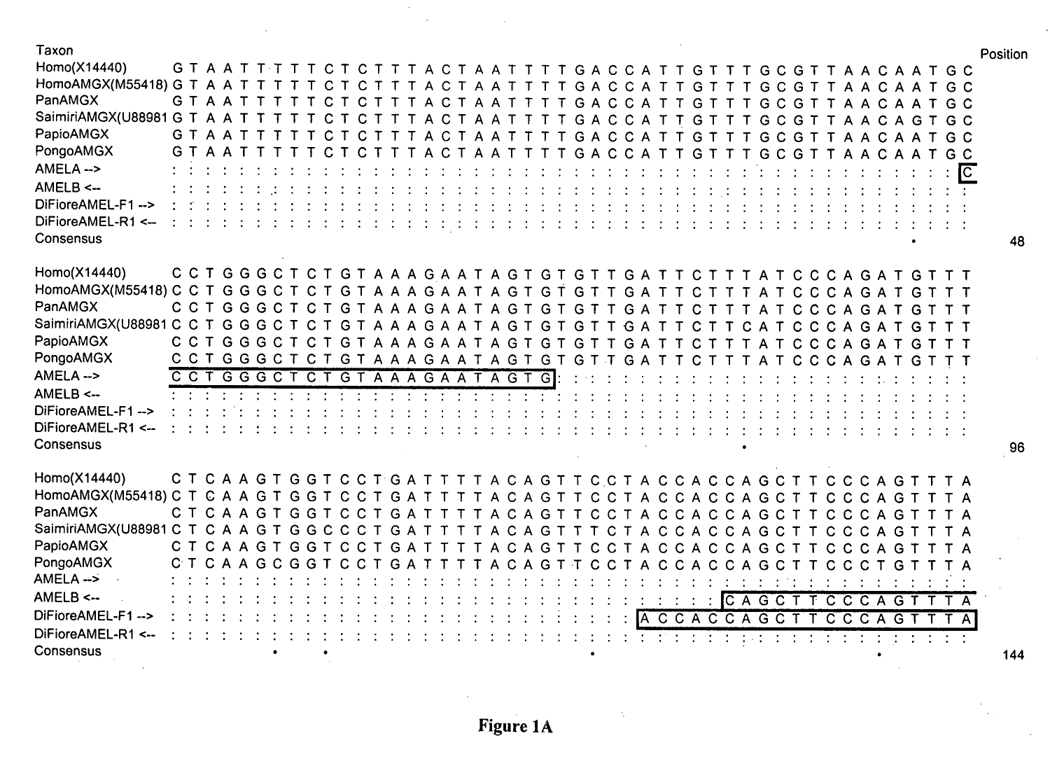

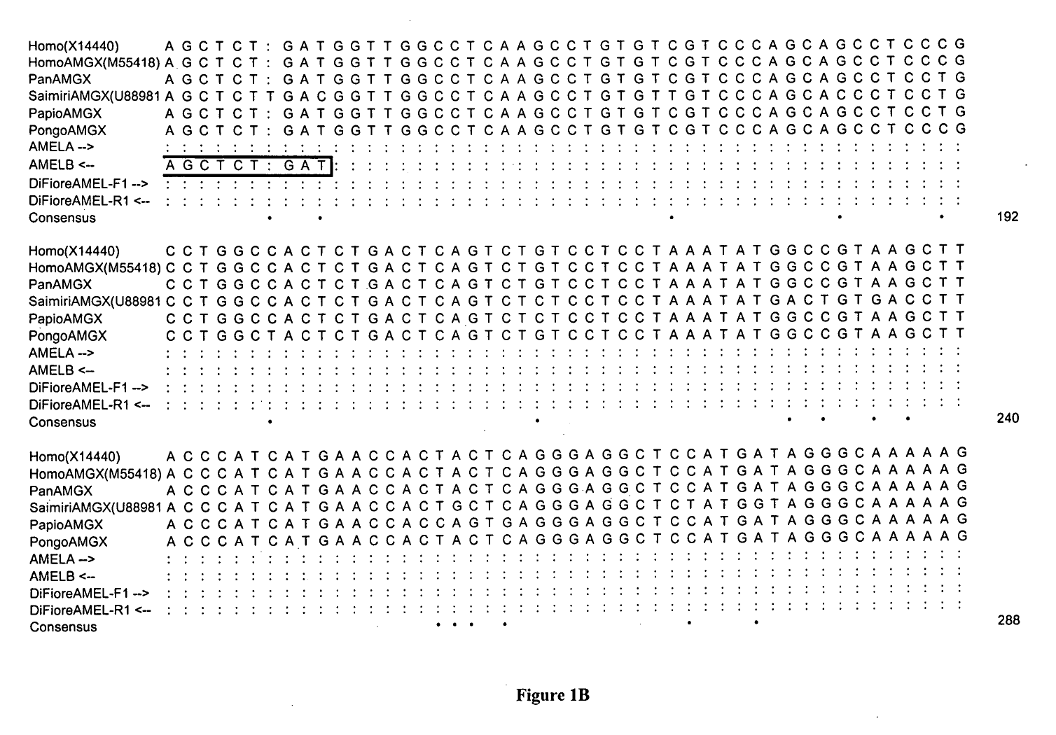

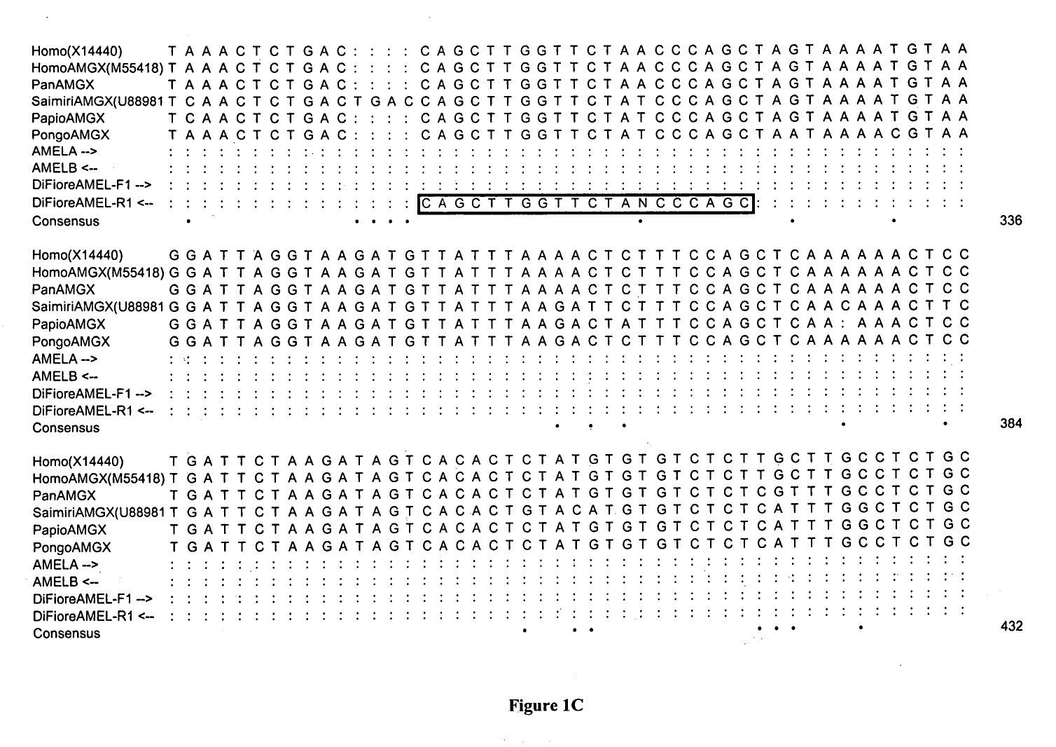

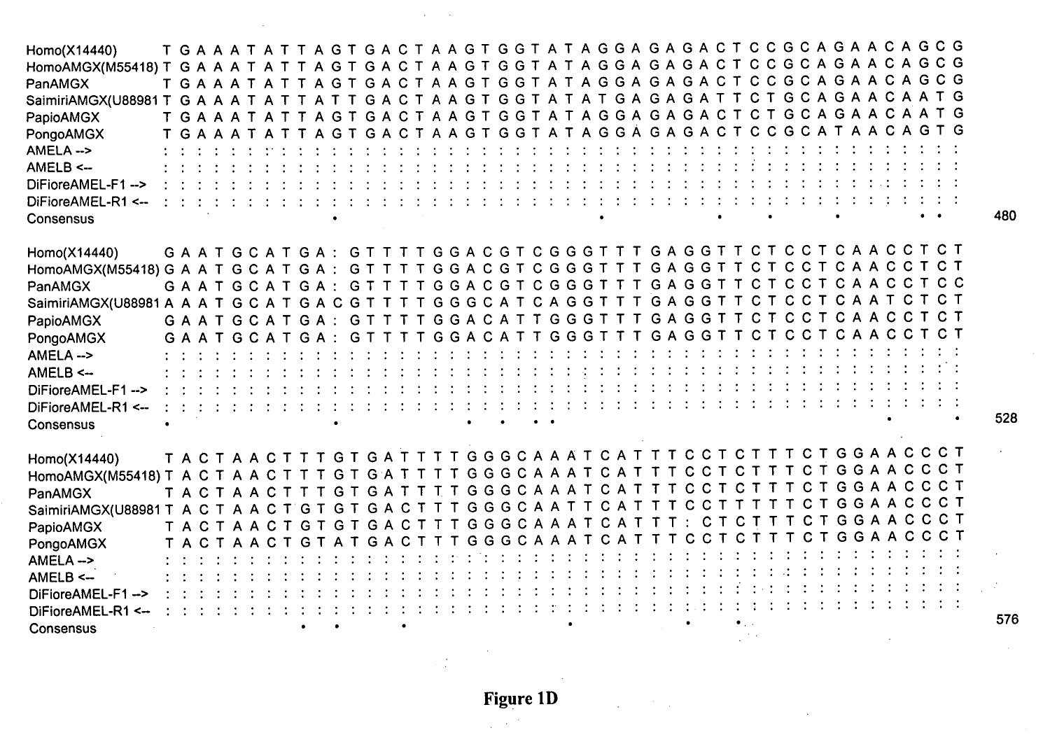

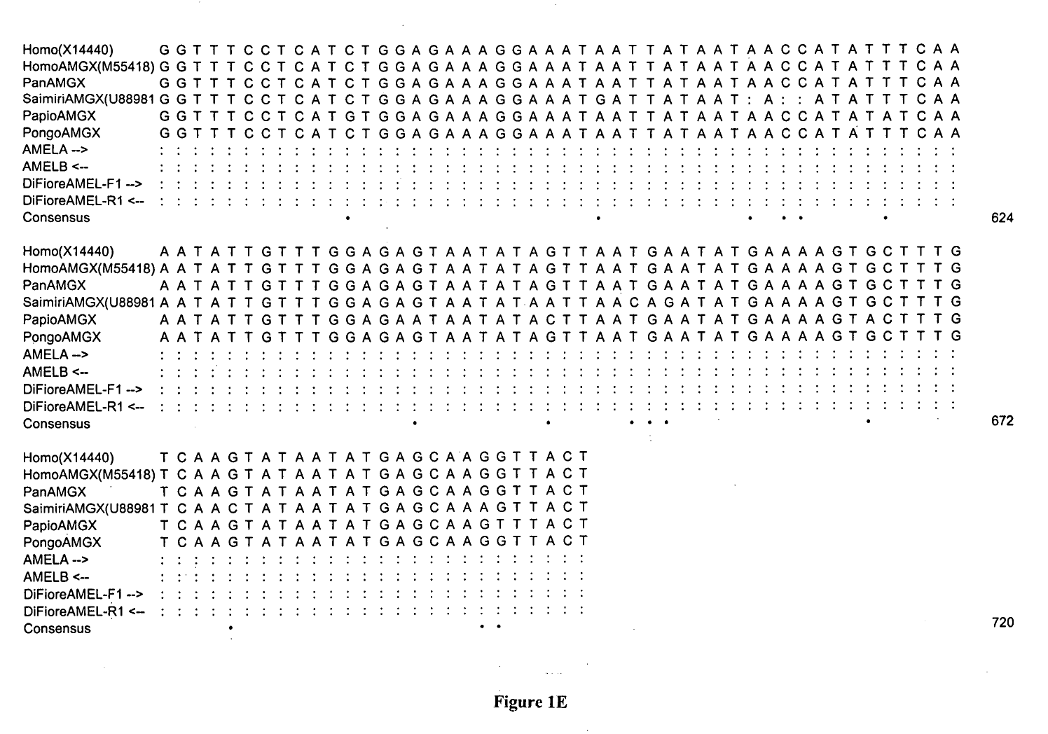

DNA sequences for the X chromosome copy of the amelogenin gene for Homo (SEQ ID NO: 1 and SEQ ID NO: 2) (humans), Pan (SEQ ID NO: 3) (chimpanzees), Saimiri (SEQ ID NO: 4) (squirrel monkeys), Papio (SEQ ID NO: 5) (baboons), and Pongo (SEQ ID NO: 6) (orangutans) downloaded from GenBank were aligned and presented in blocks of 48 bases, as shown in FIG. 1. Single black dots represent mismatches across taxon and/or primer sequences (these mismatches are what can prevent PCR primers from binding to sample DNA). The sequences of Sullivan et al.'s AMEL-A primer (SEQ ID NO: 7) and complement (SEQ ID NO: 8) of the AMEL-B primer (Sullivan et al., “A Rapid and Quantitative DNA Sex Test: Fluorescence-based PCR Analysis of X-Y Homologous Gene Amelogenin,” BioTechniques, 15:637-641 (1993), which is hereby incorporated by reference in its entirety), which were the X chromosome primers used by Steiper and Ruvolo (Steiper & Ruvolo, “Genetic Sex Identification of Orangutans.” Anthropologischer Anzeiger, 61:1-5 (2003), which is hereby incorporated by reference in its entirety), and primer SEQ ID NO-9 and the complement (SEQ ID NO: 10) of primer SEQ ID NO: 11 of the present invention are also shown.

Stretches of DNA that were at least 20 base pairs in length with no or few mismatches among taxa were identified, and primers were designed in those areas. Suitable primers were chosen by selecting primers that, for example, amplify a fragment of DNA of 150-300 base pairs, include at least 45% G+C bases (which affects, e.g., the stability of hybridization), have an estimated melting temperature of at least 58° C., and/or display minimal 3′ self-complementarity and ability to form dimers with other probes.

The primer pair SEQ ID NO: 9 and SEQ ID NO: 11 amplifies a roughly 200 base pair fragment, while amplification with SEQ ID NO: 32 and SEQ ID NO: 34 produces a roughly 165 base pair fragment, as shown in FIG. 2. A BLASTN search of the human amelogenin X and SRY fragments theoretically produced with these primer pairs against the entire human genome recovered no hits with bit scores of >66, apart from the true amelogenin X and Y and SRY genes, suggesting that these loci are, to the best of current abilities to detect them, present only as a single copy in primate genomes.

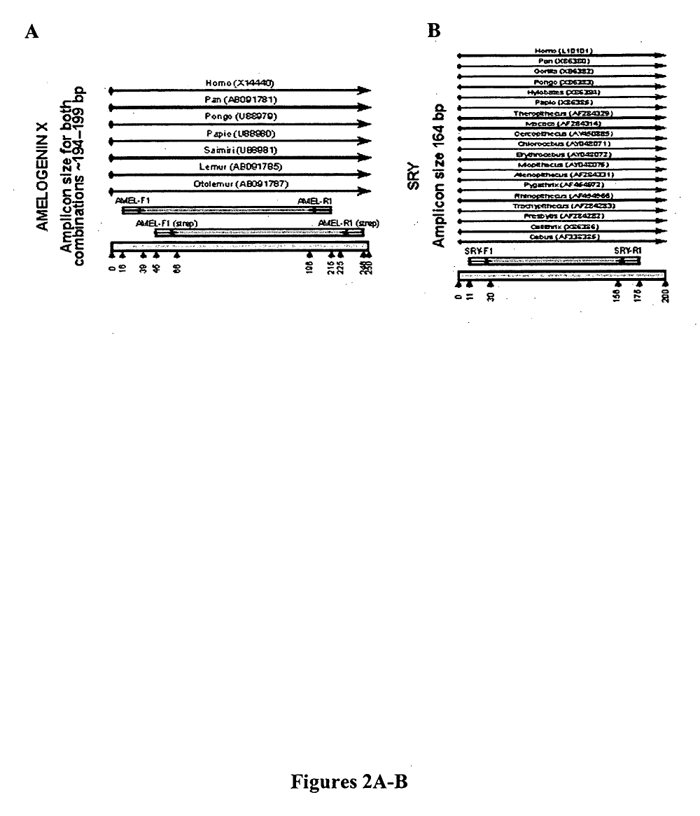

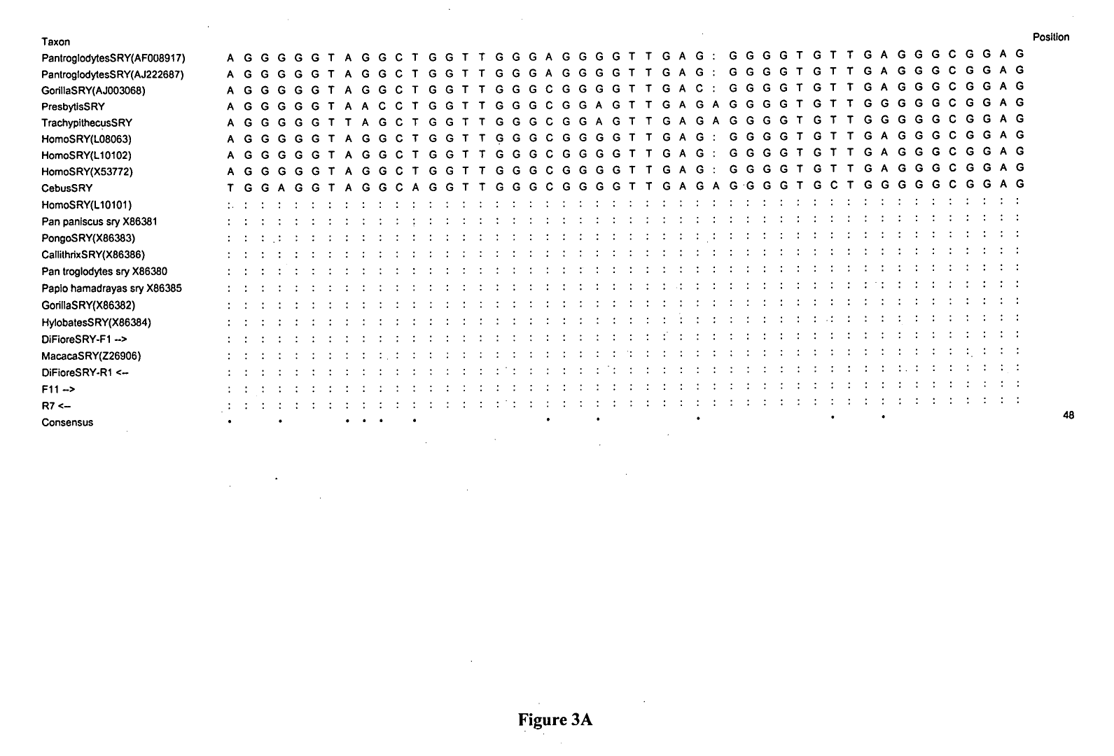

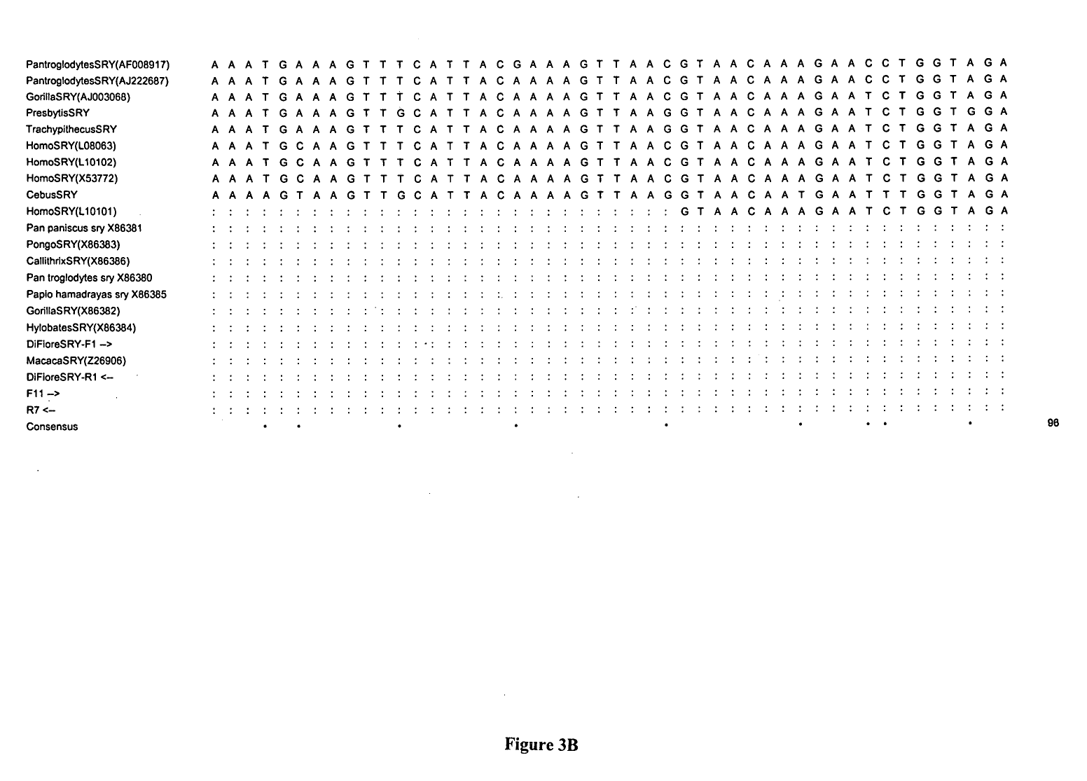

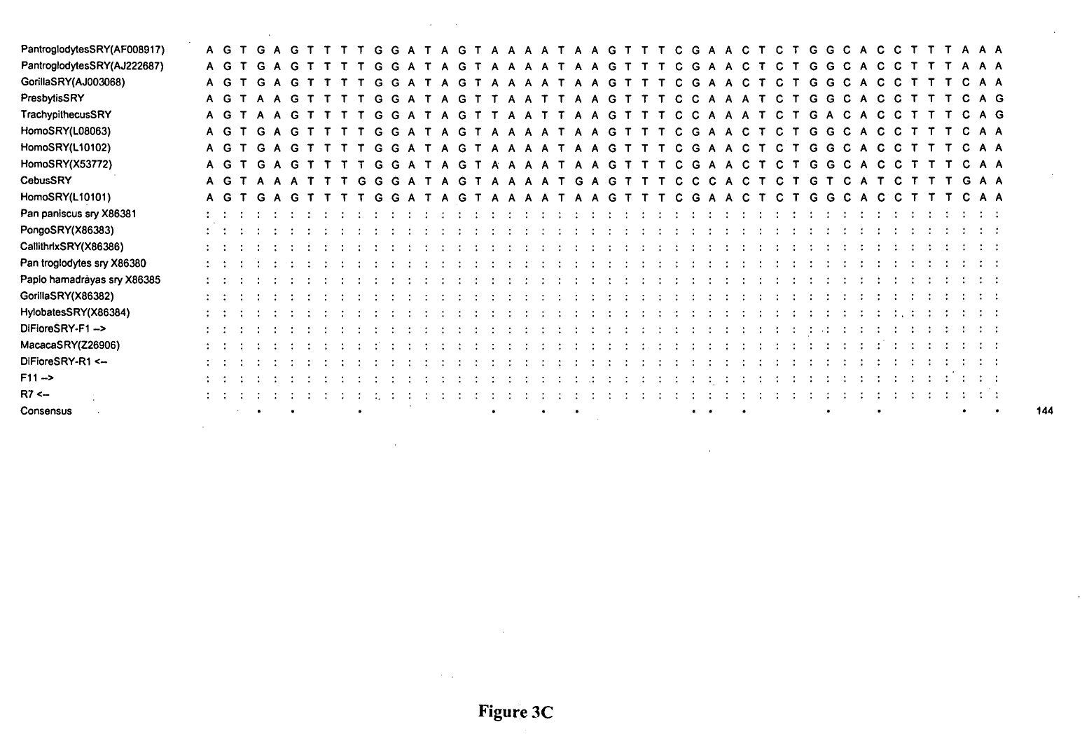

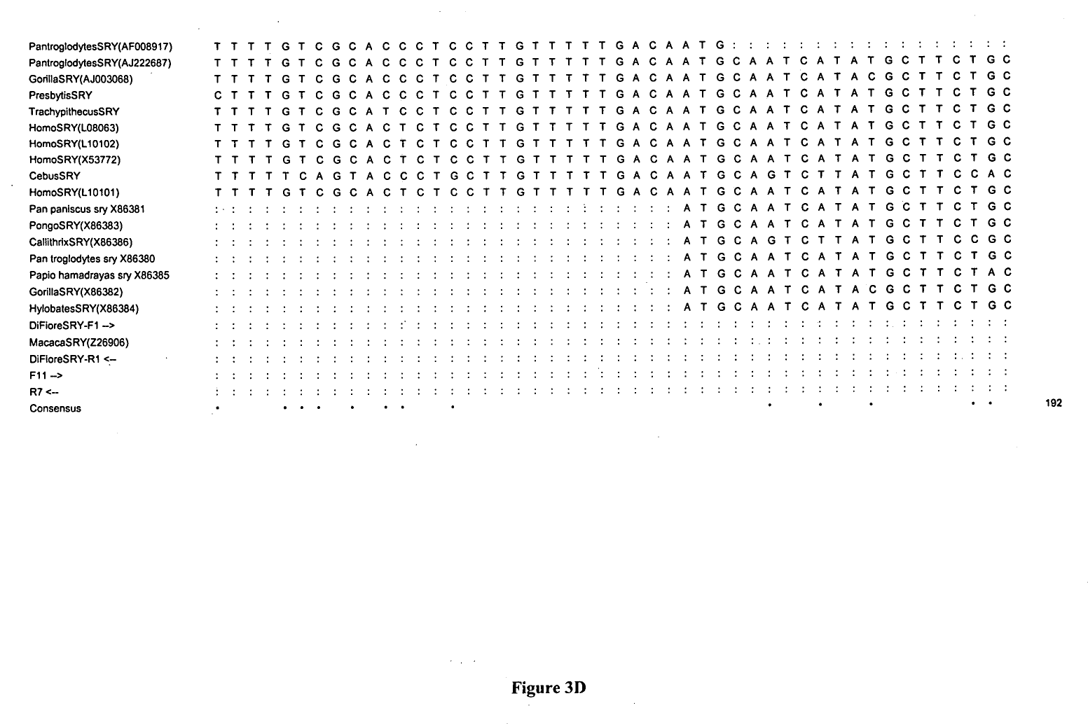

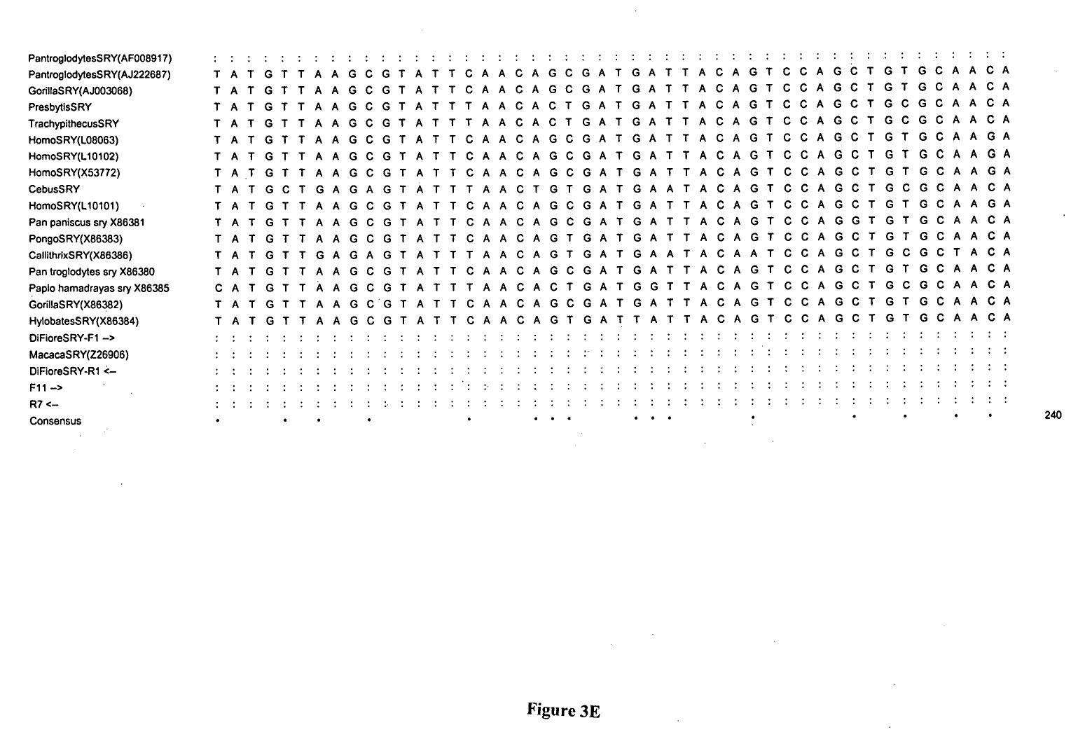

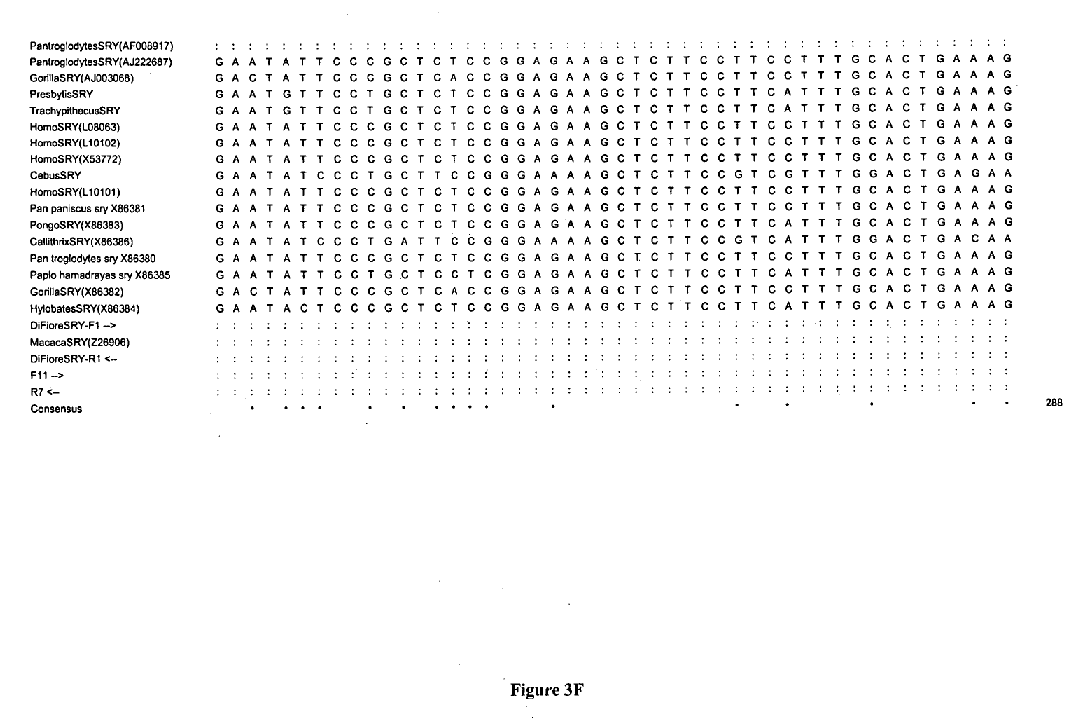

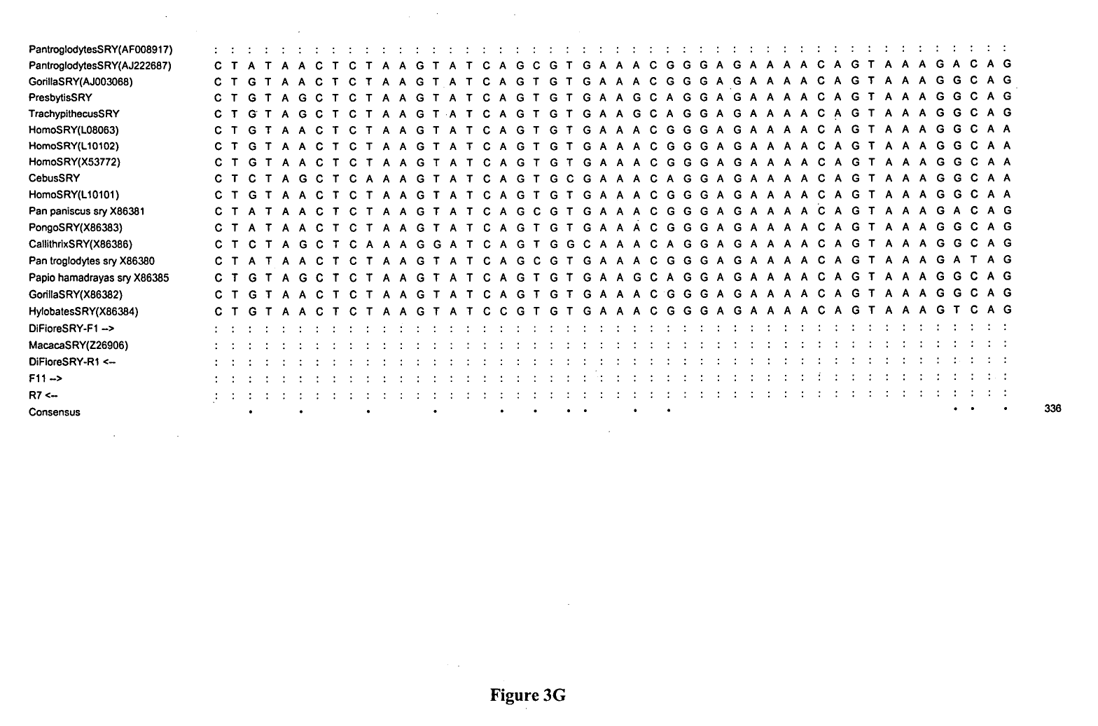

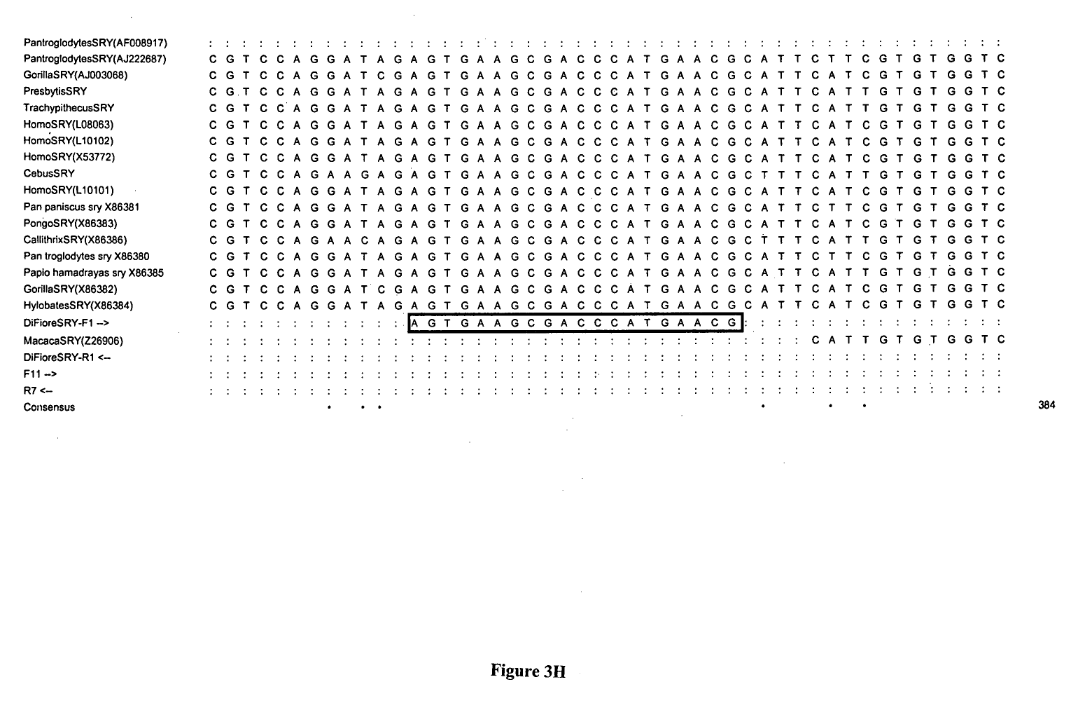

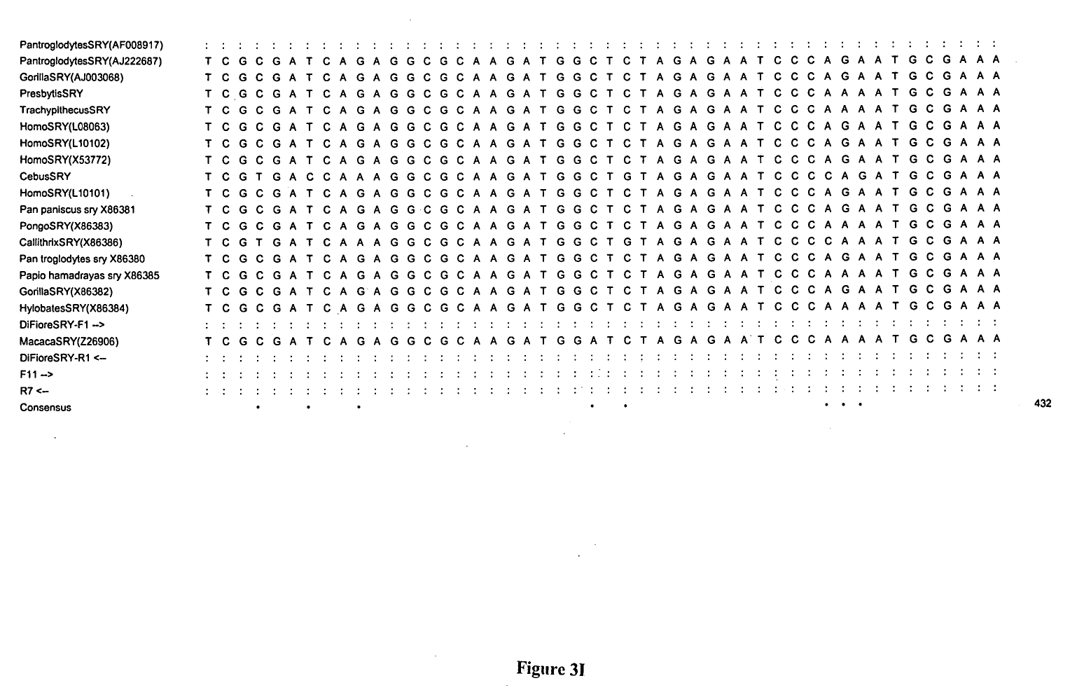

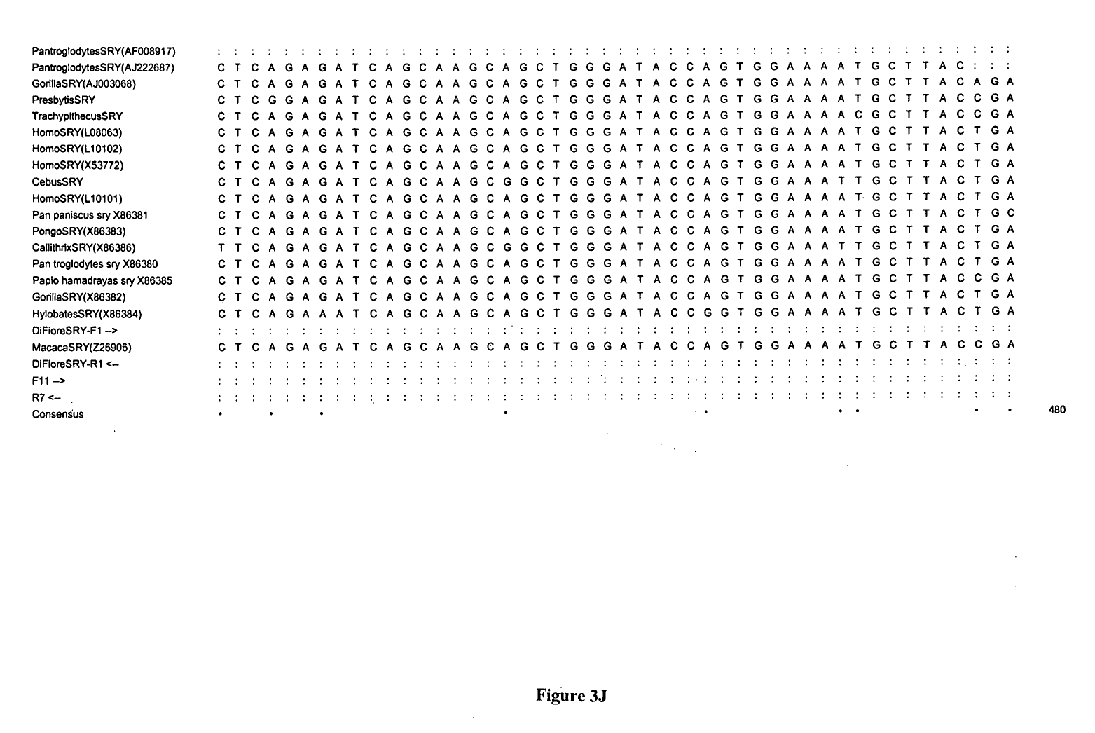

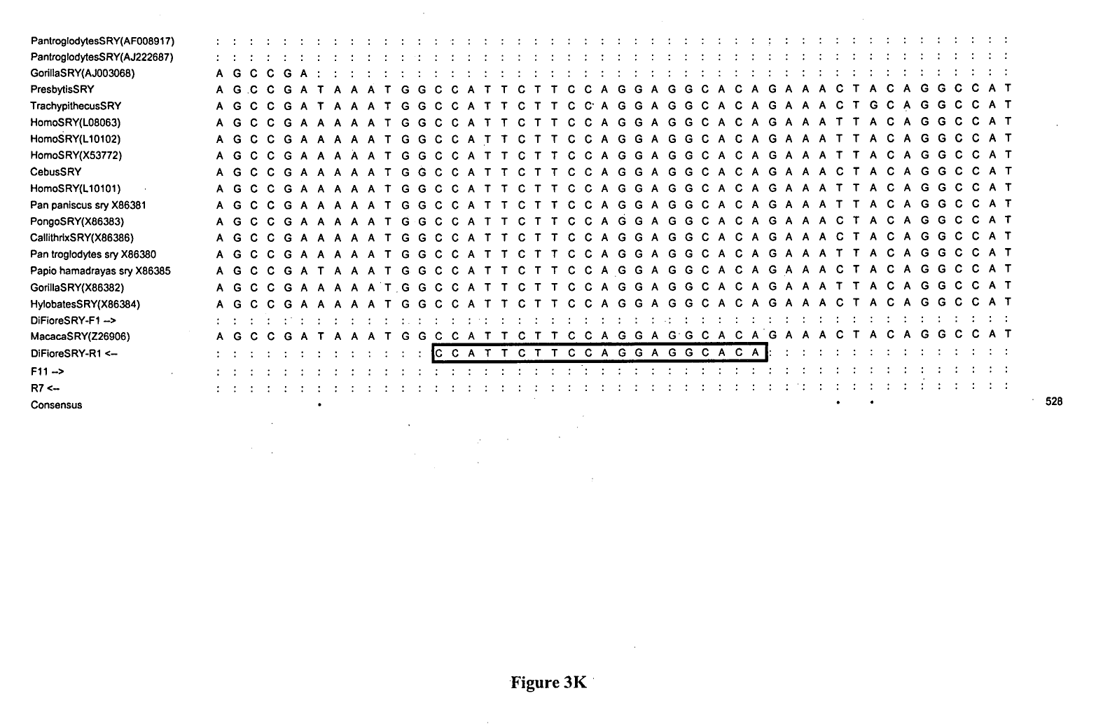

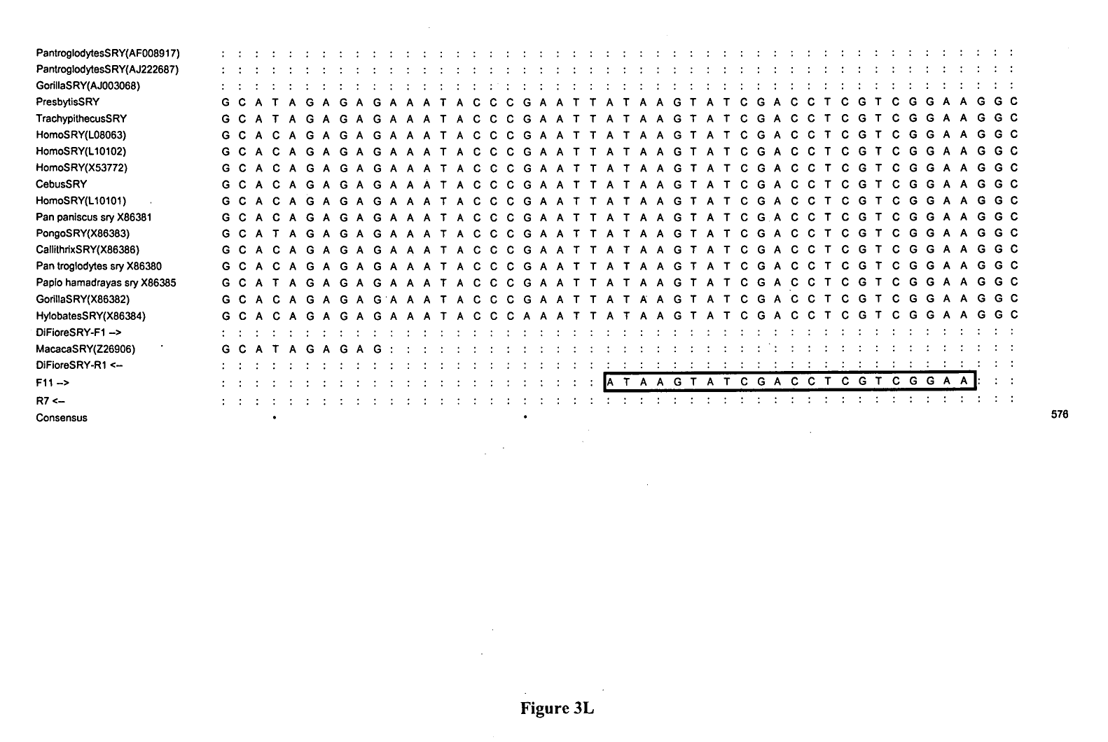

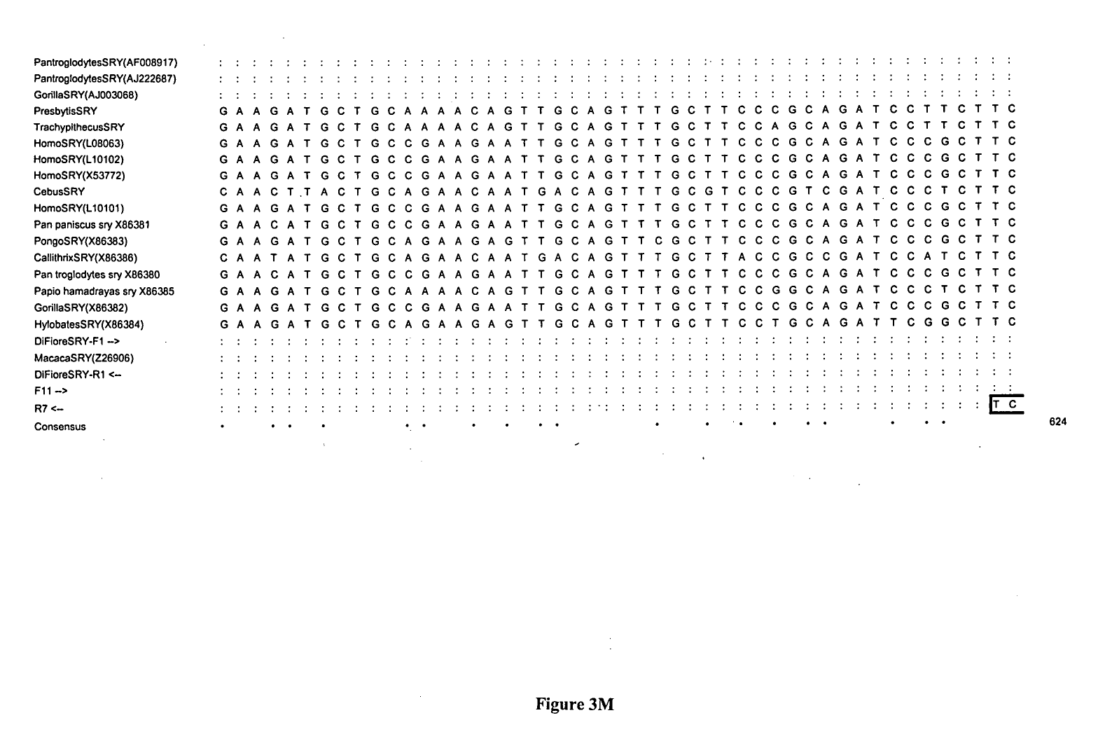

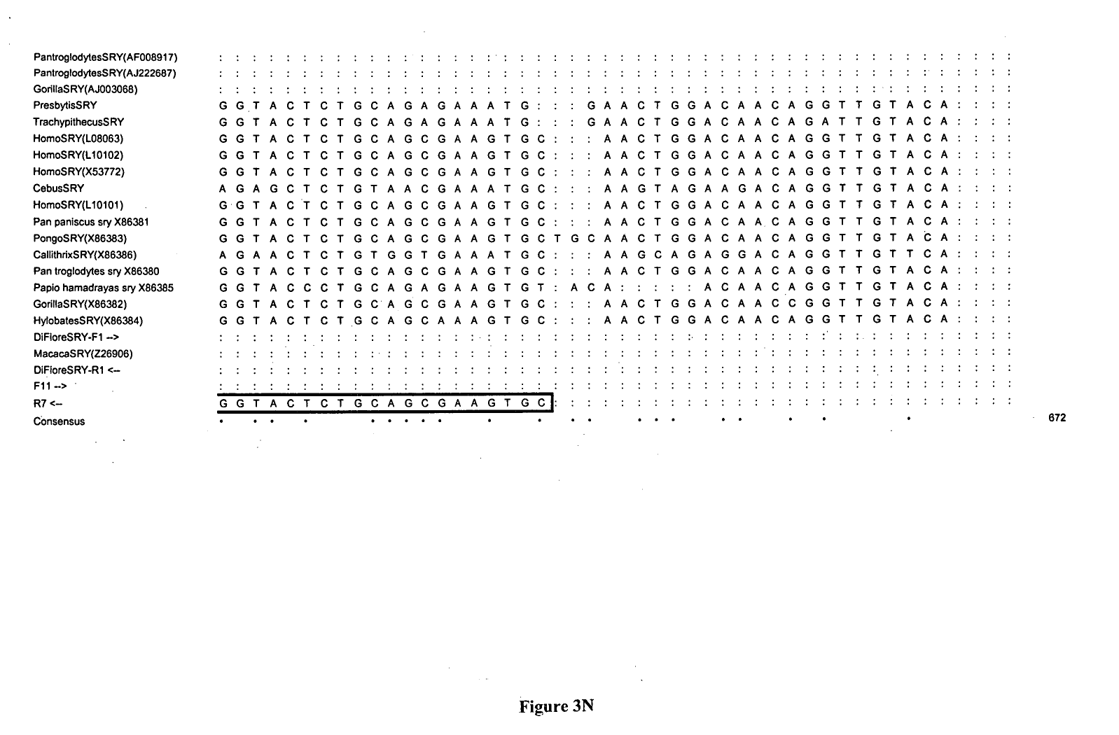

DNA sequences for the Y chromosome SRY gene for Pan troglodytes (SEQ ID NO: 12, SEQ ID NO: 13, and SEQ ID NO: 14) (common chimpanzees), Gorilla (SEQ ID NO: 15 and SEQ ID NO: 16) (gorillas), Presbytis (SEQ ID NO: 17) and Trachypithecus (SEQ ID NO: 18) (two langurs), Homo (SEQ ID NO: 19, SEQ ID NO: 20, SEQ ID NO: 21, AND SEQ ID NO: 22) (humans), Cebus (SEQ ID NO: 23) (capuchin monkeys), Pan paniscus (pygmy chimpanzees) (SEQ ID NO: 24), Pongo (orangutans) (SEQ ID NO: 25), Callithrix (SEQ ID NO 25) (marmosets), Papio hamadrayas (SEQ ID NO: 27) (baboons), Hylobates (SEQ ID NO: 28) (gibbons), and Macaca (SEQ ID NO: 29) (macaques) downloaded from GenBank were aligned and presented in blocks of 48 bases, as shown in FIG. 3. Single black dots represent mismatches across taxon and/or primer sequences. Santos et al.'s F11 primer (SEQ ID NO 29) and complement (SEQ ID NO: 31) of the R7 primer (Santos et al., “Reliability of DNA-Based Sex Tests,” Nature Genetics, 18:103 (1998), which is hereby incorporated by reference in its entirety) used by Steiper and Ruvolo (Steiper & Ruvolo, “Genetic Sex Identification of Orangutans.” Anthropologischer Anzeiger, 61:1-5 (2003), which is hereby incorporated by reference in its entirety), and primer SEQ ID NO: 32 and the complement (SEQ ID NO: 33) of primer SEQ ID NO: 34 of the present invention are also shown. It is particularly noted that Santos et al.'s R7 primer is unlikely to work across taxa, because of the numerous mismatches in the sequence.

SRY primers were designed according to the same criteria as for the amelogenin primers shown in FIG. 1. In addition, the SRY primers were designed such that, if used in a multiplex PCR assay, the SRY fragment to be amplified would be smaller than the amelogenin fragment, as a control for the PCR reaction (i.e., since smaller fragments tend to amplify more readily, if the larger X fragment amplified from a sample but the smaller Y fragment did not, it is more certain that the sample is from a female).

### Example 2

### Collection Of Biological Samples

Genomic DNA for a variety of platyrrhine primates (New World monkeys) was extracted from various field-collected source material (blood, tissue, hair, and feces) using commercially available DNA extraction kits (e.g., QIAgen™ DNeasy Tissue Kits and QIAmp™ DNA Stool Mini Kits). DNA samples for the remaining taxa were provided by colleagues in the Molecular Anthropology Laboratory at New York University. In all, 77 samples from 38 primate genera were examined, as shown in Table 1. The Lagothrix fecal sample was desiccated in silica gel, while Ateles fecal samples were collected and stored in RNAlater™ (Ambion). The assay was also tested and worked on Papio sp. fecal samples stored in RNAlater™ (Ambion) and on Leontopithecus rosalia hair samples that were collected and stored in plastic envelopes with no desiccating agent or preservative.

TABLE 1
Primate samples sex-typed

| | | | | | |
|---|---|---|---|---|---|
| Taxonomic Group/ | | Sample | | Reported | Assigned |
| Genus and Species | Source^{1,2} | Type³ | Sample ID | Sex | Sex |

STREPSIRHINI
Lemuroidea

| | | | | |
|---|---|---|---|---|
| Lemur catta | D Wildman | DNA | Male | Male |
| Mirza coquereli | D Wildman | DNA | Unknown | Male |
| Otolemur crassicaudatus | D Wildman | DNA | Female | Female |
| Daubentonia | D Wildman | DNA | Male | Male |
| madagascariensis | | | | |

HAPLORHINI
Tarsioidea

| | | | | |
|---|---|---|---|---|
| Tarsiidae | | | | |
| Tarsius syrichta | D Wildman | DNA | Female | Female |

Platyrrhini

| | | | | | |
|---|---|---|---|---|---|
| Cebidae | | | | | |
| Cebus albifrons | A Di Fiore^{a} | Tissue | MF7 | Unknown | Male |
| Saimiri sciureus | A Di Fiore^{a} | Tissue | MF6 | Unknown | Female |
| Saimiri sciureus | A Di Fiore | Blood | | Male | Male |
| Aotus vociferans | A Di Fiore^{a} | Tissue | MF20 | Unknown | Female |
| Aotus lemurinus | T Disotell | DNA | | Female | Female |
| Saguinus oedipus | T Tosi | Blood | SAG1 | Male | Male |
| Leontopithecus rosalia | J Dietz^{b} | Hair | 686H | Male | Male |
| Leontopithecus rosalia | J Dietz^{b} | Hair | 601H | Female | Female |
| Cebuella pygmaea | A Di Fiore^{a} | Tissue | T99 | Unknown | Male |
| Pithecidae | | | | | |
| Pithecia pithecia | R Araya | Tissue | Miles | Male | Male |
| Chiropotes satanus | R Araya | Tissue | | Unknown | Female |
| Callicebus discolor | A Di Fiore^{a} | Tissue | CD1 | Male | Male |
| Callicebus donacophilus | C Lehn | Tissue | 961043 | Male | Male |
| Atelidae | | | | | |
| Lagothrix lagotricha | A Di Fiore^{a} | Tissue | T30 | Male | Male |
| Lagothrix lagotricha | A Di Fiore^{a} | Tissue | T13 | Female | Female |
| Lagothrix lagotricha | A Di Fiore^{a} | Tissue | T2 | Female | Female |
| Lagothrix lagotricha | A Di Fiore^{a} | Tissue | LL2000-10 | Unknown | Female |
| Lagothrix lagotricha | A Di Fiore^{a} | Feces | LL2000-F12 | Male | Male |
| Ateles belzebuth | A Di Fiore^{a} | Tissue | Omaca1 | Female | Female |
| Ateles belzebuth | A Di Fiore^{a} | Tissue | MF22 | Male | Male |
| Ateles belzebuth | S Spehar^{a} | Feces | Oko | Male | Male |
| Ateles belzebuth | S Spehar^{a} | Feces | Kuraka | Female | Female |
| Ateles belzebuth | S Spehar^{a} | Feces | Toma | Female | Female |
| Ateles belzebuth | S Spehar^{a} | Feces | Oso | Male | Male |
| Ateles belzebuth | S Spehar^{a} | Feces | Kaya | Female | Female |
| Alouatta seniculus | R Rudran^{c} | Blood | AS1 | Male | Male |
| Alouatta seniculus | R Rudran^{c} | Blood | AS2 | Female | Female |
| Alouatta seniculus | R Rudran^{c} | Blood | AS3 | Female | Female |
| Alouatta seniculus | R Rudran^{c} | Blood | AS4 | Male | Male |
| Alouatta seniculus | A Di Fiore^{a} | Tissue | Omaca2 | Male | Male |
| Alouatta seniculus | A Di Fiore^{a} | Tissue | MF21 | Male | Male |

Cercopithecoidea

| | | | | | |
|---|---|---|---|---|---|
| Cercopithecinae | | | | | |
| Macaca sp | T Disotell | DNA | | Male | Male |
| Cercopithecus ascanius | T Tosi | DNA | 41137B | Male | Male |
| Cercopithecus nictitans | R Rauum | DNA | OR1622 | Female | Female |
| Cercopithecis sp | T Tosi | DNA | 100088 | Female | Female |
| Cercocebus torquatus | R Rauum | DNA | OR538 | Male | Male |
| Chlorocebus aethiops | T Tosi | DNA | 1149 | Female | Female |
| Chlorocebus aethiops | R Rauum | DNA | VE98007 | Male | Male |
| Lophocebus aterrimus | R Rauum | DNA | | Male | Male |
| Allenopithecus nigroviridis | R Rauum | DNA | | Male | Male |
| Erythrocebus patas | R Rauum | DNA | | Male | Male |
| Papio sp | A Burrell | DNA | SWF 18737 | Male | Male |
| Papio sp | A Burrell | DNA | SWF 18736 | Female | Female |
| Papio sp | R Palombit^{d} | Feces | | Male | Male |
| Papio sp | R Palombit^{d} | Feces | | Female | Female |
| Theropithecus gelada | R Rauum | DNA | 8910961 | Male | Male |
| Mandrillus leucophaeus | R Raaum | DNA | OR919 | Female | Female |
| Mandrillus sphinx | S Clifford^{e} | DNA | | Male | Male |
| Mandrillus sphinx | S Clifford^{e} | DNA | | Female | Female |
| Colobinae | | | | | |
| Procolobus badius | T Pope | DNA | | Male | Male |
| Colobus guereza | N Ting | DNA | | Unknown | Female |
| Presbytis melalophos | R Rauum | DNA | DJ30 | Male | Male |
| Nasalis larvatus | R Raaum | DNA | | Unknown | Male |
| Pygathrix nemaeus | R Raaum | DNA | OR615 | Male | Male |

Hominoidea

| | | | | | |
|---|---|---|---|---|---|
| Homo sapiens | T Disotell^{f} | DNA | HS1 | Male | Male |
| Homo sapiens | T Disotell^{f} | DNA | HS2 | Female | Female |
| Homo sapiens | T Disotell^{f} | DNA | HS3 | Female | Female |
| Pan troglodytes | R Raaum | DNA | NA03448A | Unknown | Male |
| Pongo pygmaeus | R Raaum | DNA | NA04272 | Unknown | Male |
| Pongo pygmaeus | T Disotell | DNA | O-1 | Unknown | Female |
| Pongo pygmaeus | T Disotell | DNA | O-2 | Unknown | Male |
| Pongo pygmaeus | T Disotell | DNA | O-3 | Unknown | Female |
| Pongo pygmaeus | T Disotell | DNA | O-4 | Unknown | Male |
| Gorilla gorilla | J Satkowski | DNA | GG1 | Female | Female |
| Gorilla gorilla | R Raaum | DNA | NG05251B | Unknown | Female |
| Gorilla gorilla | T Disotell | DNA | G-1 | Unknown | Female |
| Gorilla gorilla | T Disotell | DNA | G-2 | Unknown | Female |
| Gorilla gorilla | T Disotell | DNA | G-3 | Unknown | Male |
| Gorilla gorilla | T Disotell | DNA | G-4 | Unknown | Female |
| Hylobates agilis | R Raaum | DNA | 0291 | Unknown | Male |
| Symphalangus syndactylus | R Raaum | DNA | OR790 | Male | Male |
| Symphalangus syndactylus | S Lappan | DNA | SL1 | Male | Male |

¹Researchers providing the source material or DNA used. Contributing organizations for some of these source materials include the Bronx Zoo-Wildlife Conservation Society, the Center for Reproduction of Endangered Species, the Cheyene Mountain Zoo, the Duke University Primate Center, Harvard University, the Louisiana Purchase Zoo, New York University, the Oakland Zoo, the Southwest Foundation for Primate Research, and the State University of New York at Albany.
²The source country is noted if the sample is known to be from a wild population. ^{a}Ecuador ^{b}Brazil ^{c}Venzuela ^{d}Kenya ^{e}Gabon ^{f}Anonymous - US
³Where “DNA” is listed as the sample type, high quality genomic DNA extracted from blood, tissue, or cell culture was provided by the researcher listed. For all other sample types, genomic DNA was extracted from the material listed using commercially available DNA extraction kits (QIAgen ™).

### Example 3

### Multiplex PCR With Anthropoid Primers

Multiplex PCR with primers SEQ ID NO: 9, SEQ ID NO: 11, SEQ ID NO: 32, and SEQ ID NO: 34 provides an unambiguous and rapid sex determination assay that is broadly applicable across anthropoid primates.

Varying subsets of the samples listed in Table 1 amplified relatively cleanly in a range of early PCR trials during which different annealing temperatures and primer and magnesium concentrations were experimented with, as well as with several alternate primer oligonucleotides slightly different from those described herein. Throughout all of these optimization trials, the basic assay gave consistently good results with respect to sex determination.

Following optimization, the full set of samples was run. The multiplex PCR mix consisted of 2.5 μL of Mg-free 10× Promega™ PCR Buffer, 2.0 μL of 10 mM dNTP mix (2.5 mM each dNTP, Promega™), 1.5 μL of 25 mM MgCl₂ (Promega™), 1 μL of 100× BSA (10 mg/mL) (New England Biolabs or Promega™), 0.8 μL of each.primer at 10 μM concentration, 1.5 Units Promega™ Taq DNA polymerase, ˜25 to 100 ng DNA template, plus ddH₂O up to a total volume of 25 μL. The cycling profile for the reaction, run on a variety of thermal cyclers (e.g, MJ Research PTC-100, BioRad iCycler, Perkin-Elmer 9600), included an initial denaturing step at 94° C. for 2 minutes; 40 to 45 cycles of 94° C. for 30 seconds, 58° C. for 30 seconds, and 72° C. for 30 seconds; and a final extension at 72° C. for 5 minutes. For each PCR, 40 cycles were used for high quality DNA templates, while either 40 or 45 cycles were used for extracts from hair and feces. Reactions were set up in a UV-irradiated HEPA-filter equipped PCR workstation using aerosol barrier tips for liquid reagent and DNA handling in order to minimize the likelihood of contamination with exogenous DNA, and negative controls were run for all reactions.

PCR products were separated by benchtop gel electrophoresis in 8% acrylamide minigels (600 μL 10× TBE, 1200 μL 40% 29:1 acrylamide:bis-acrylamide gel stock, and ddH₂O up to a total volume of 6 ml; plus 60 μL 10% ammonium persulfate and 6 μL TEMED to catalyze polymerization). Gels were run at ˜80 to 85V for 90 to 120 minutes and visualized by UV light following staining with ethidium bromide.

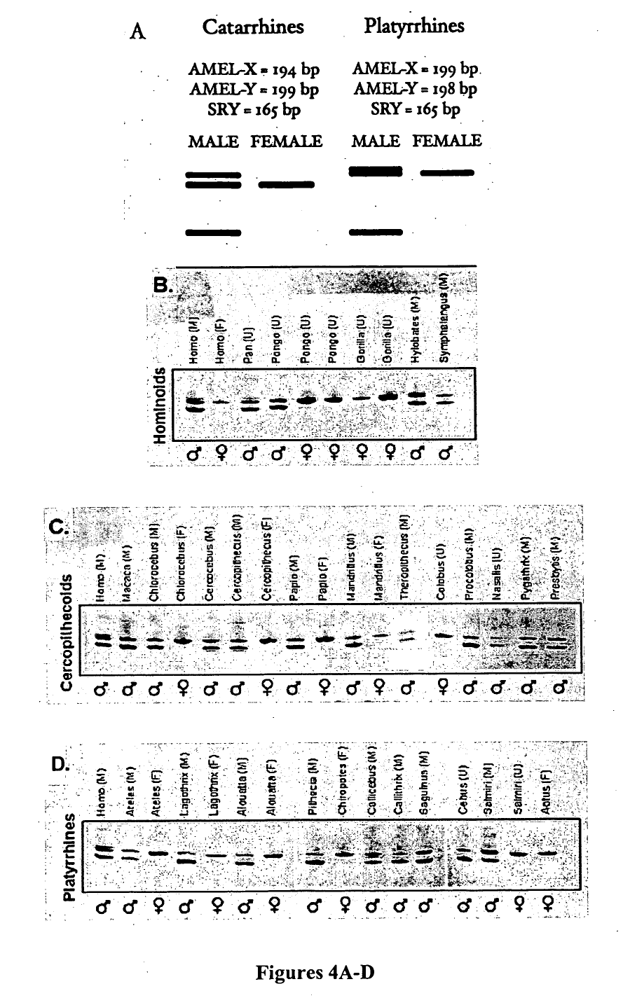

FIG. 4A outlines the expected results of the multiplex PCR assay, and FIGS. 4B through 4D demonstrate its efficacy for sexing high quality DNA of taxa from across anthropoid primates, including those for which the simple amelogenin method of sex determination (Sullivan et al., “A Rapid and Quantitative DNA Sex Test: Fluorescence-based PCR Analysis of X-Y Homologous Gene Amelogenin,” BioTechniques, 15:637-641 (1993); Mannucci et al., “Forensic Application of a Rapid and Quantitative DNA Sex Test by Amplification of the X-Y Homologous Gene Amelogenin,” Int. J. Legal Med., 106:190-193 (1994), which are hereby incorporated by reference in their entirety) has proven ineffective. Every anthropoid DNA sample from 33 different genera was easily scored as female or male based on the presence or absence of bands of the appropriate size following amplification, and all samples from individuals of known sex (52 individual samples from 25 genera) were typed correctly, as shown in Table 1.

Moreover, the assay performed well on DNA samples extracted from feces or hair of known-sex individuals from the four anthropoid genera (Lagothrix, Ateles, Leontopithecus, and Papio) in which they were tested, as shown in FIG. 5, although multiple reactions were sometimes required before the samples could be successfully typed.

### Example 4

### Development Of Strepsirhine-Specific Primers

In the four strepsirhine taxa tested, amplification of the X fragment using the primers SEQ ID NO: 9 and SEQ ID NO: 11 was either very weak or not observed at all, although the SRY band amplified strongly in known male samples. Thus, strepsirhine-specific amelogenin primers (SEQ ID NO: 37 and SEQ ID NO: 39) were designed based upon published sequence data for Lemur catta and Otolemur garnetti.

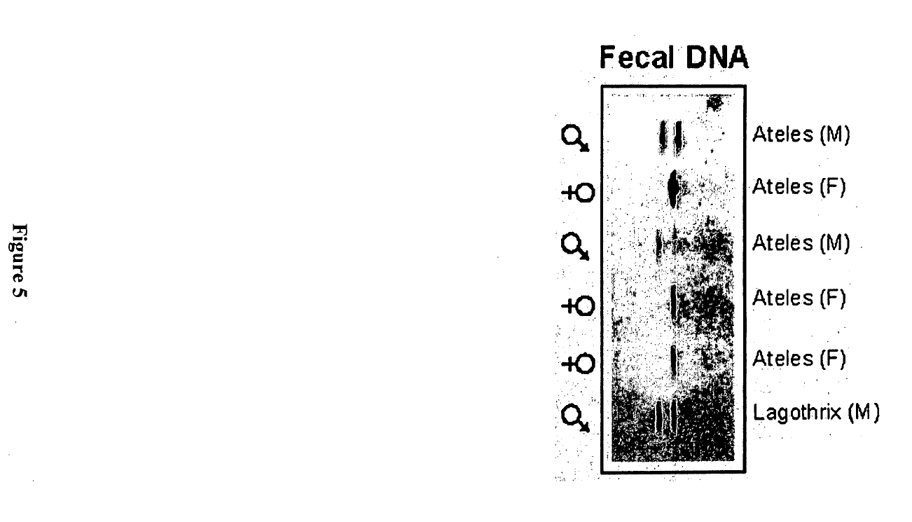

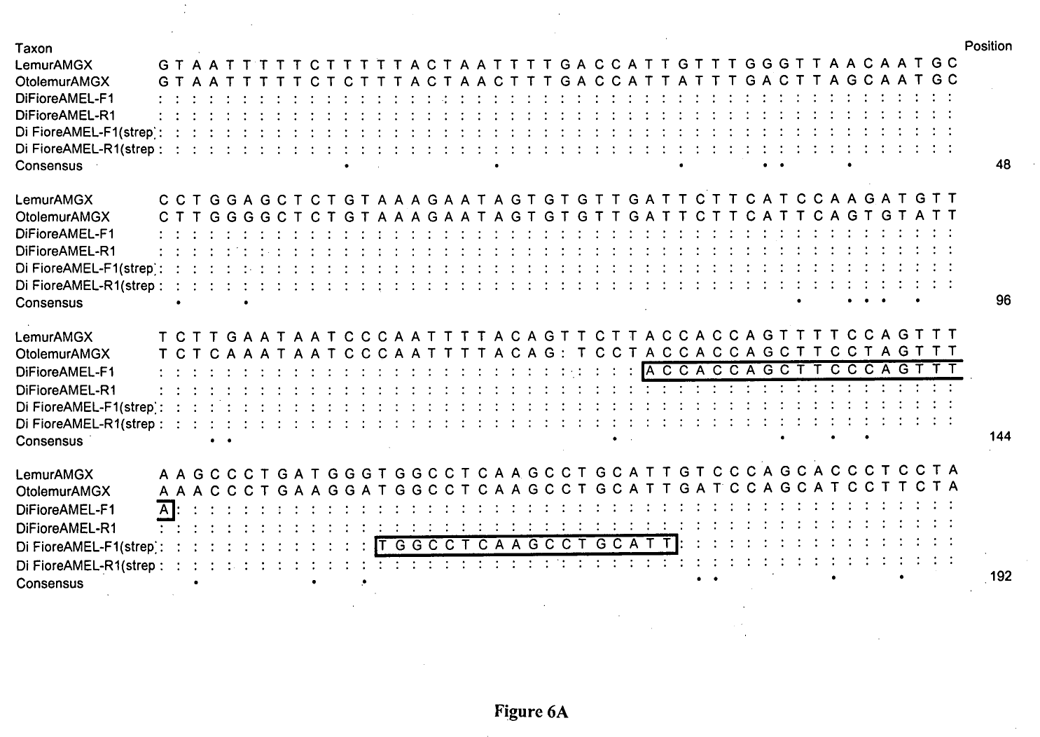

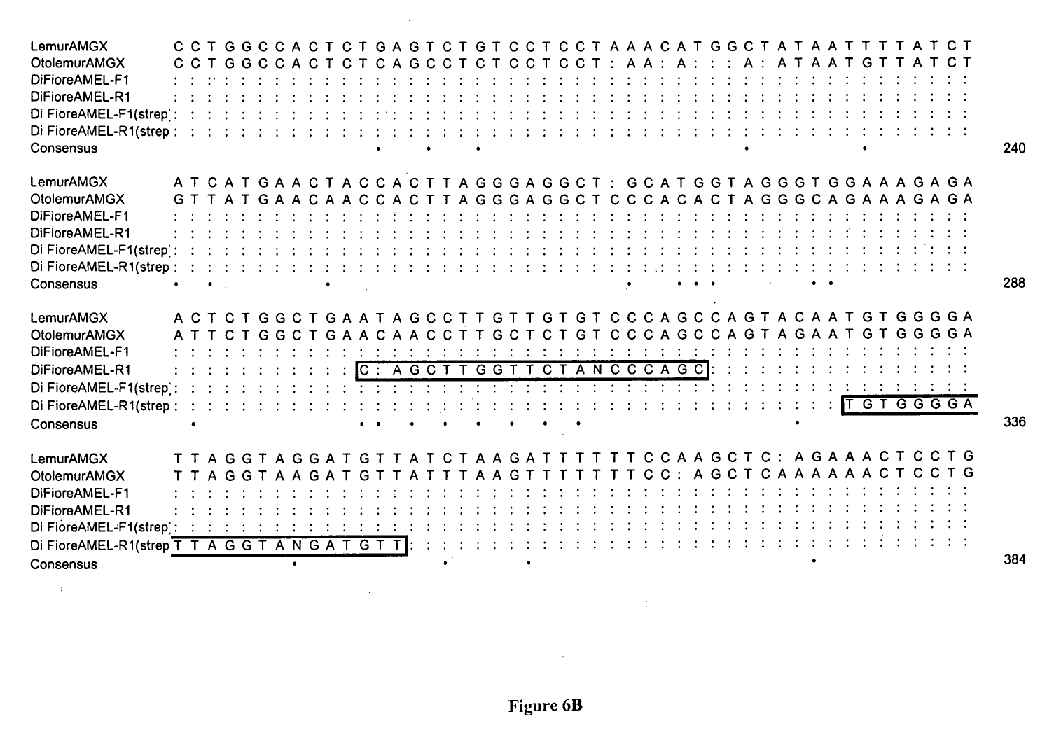

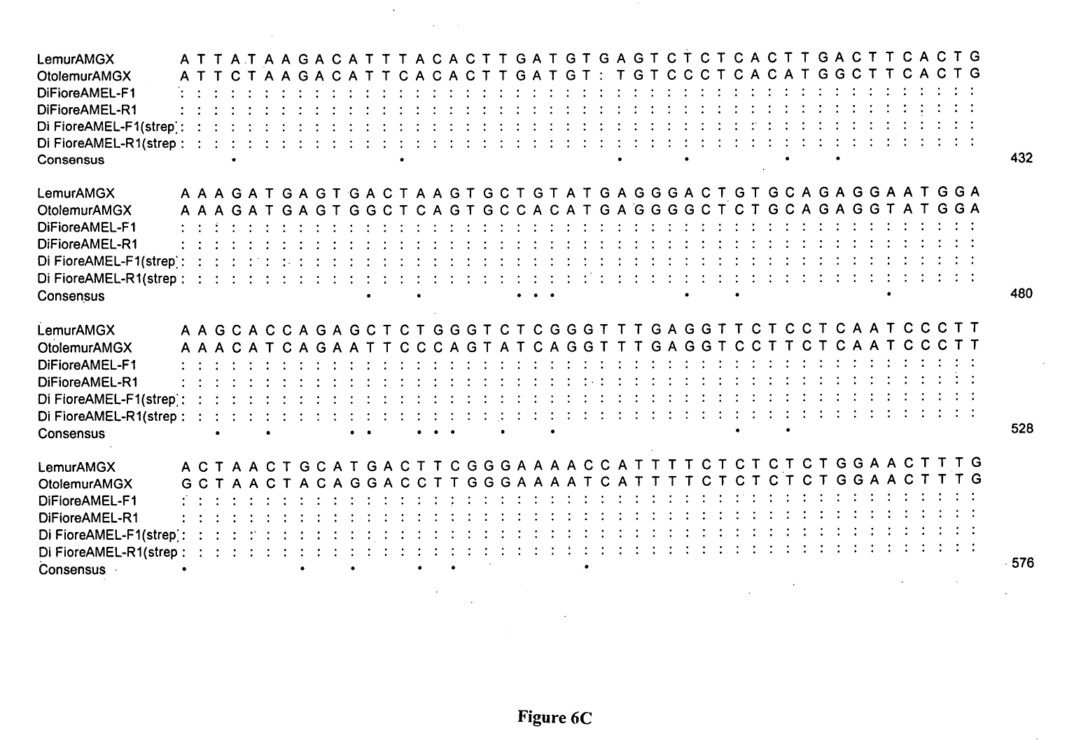

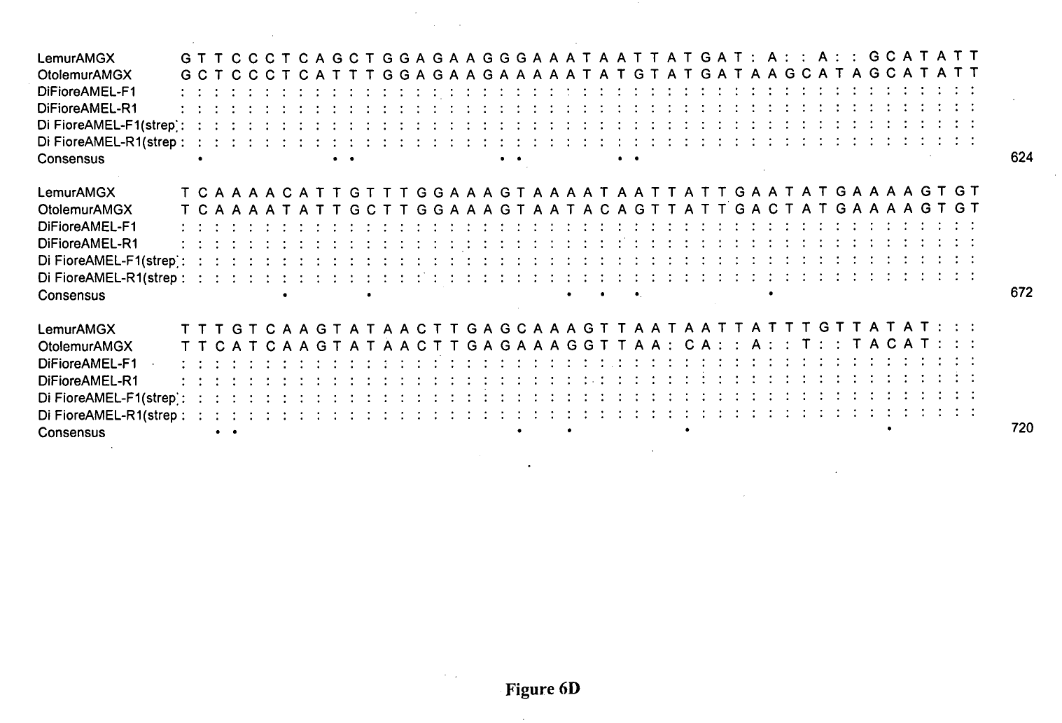

DNA sequences for the X chromosome copy of the amelogenin gene for Lemur (SEQ ID NO: 35) and Otolemur (SEQ ID NO: 36) downloaded from GenBank were aligned and presented in blocks of 48 bases, as shown in FIG. 6. Single black dots represent mismatches among taxes (these mismatches are what can prevent PCR primers from binding to sample DNA). Anthropoid amelogenin primer SEQ ID NO: 9 and the complement (SEQ ID NO: 10) of primer SEQ ID NO: 11 of the present invention, and strepsirhine amelogenin primer SEQ ID NO: 37 and the complement (SEQ ID NO: 38) of primer SEQ ID NO: 39 of the present invention, are also shown. FIG. 6 shows why different amelogenin primers are used for Strepsirhines versus Anthropoids. In particular, there are many mismatches among strepsirhine taxa in the anthropoid primer regions. The strepsirhine primers are staggered roughly 10 base pairs from the anthropoid primers to produce comparably sized PCR fragments in both groups of primates.

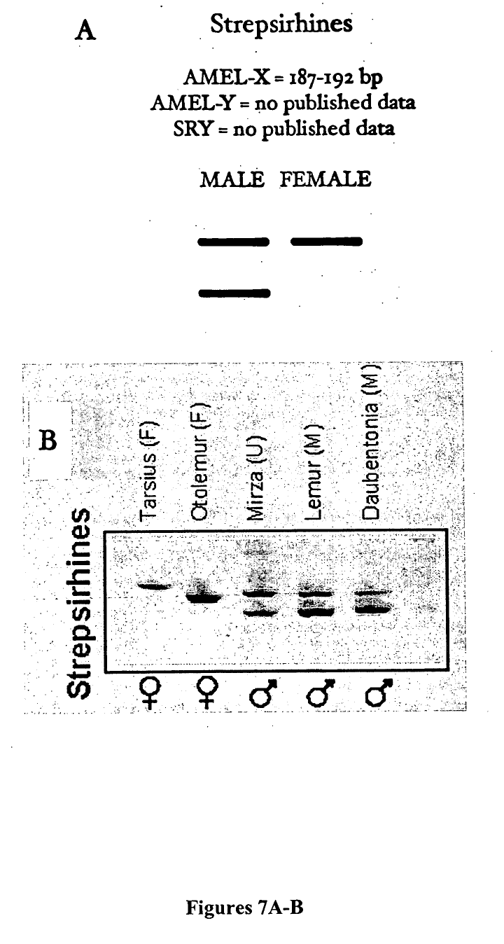

The strepsirhine primers amplify a nearly identically-sized X chromosome fragment to that produced by SEQ ID NO: 9 and SEQ ID NO: 11 in anthropoids, and, when multiplexed with SEQ ID NO: 32 and SEQ ID NO: 34 using the same PCR mix and thermal cycling conditions described in Example 3 above, appear to provide a comparable sex assignment test, as demonstrated in FIG. 7.

### Example 5

### Predicted Efficacy Of Strepsirhine-Specific Primers On Tarsius Samples

Both the anthropoid and strepsirhine primer combinations were tested on a single female tarsier (Haplorrhini: Tarsidae) sample. While the former combination produced no amplicon, the latter, as shown in FIG. 7, lane 1, yielded a single-banded product ˜20 base pairs larger than the known amelogenin X fragment for both anthropoids and strepsirhines, and no fragment near the size expected for the SRY amplicon. Thus, although no known male Tarsius sample was available for testing, it is expected that the strepsirhine-specific primers may be used to sex this genus as well.

### Example 6

### PCR with Fluorescent-Labeled Primers

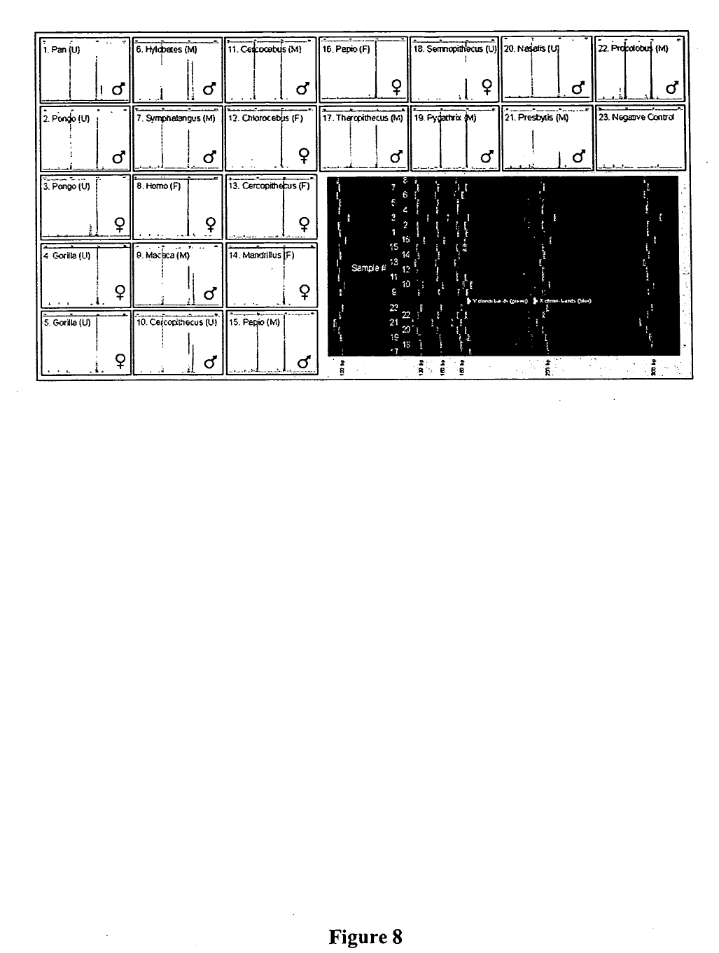

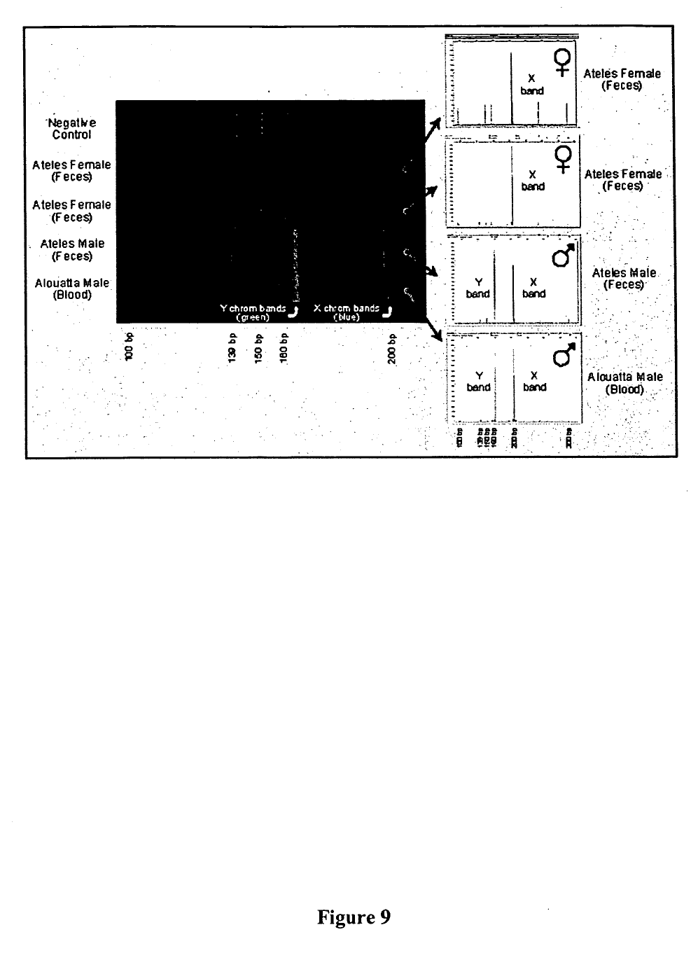

The anthropoid primer combination was tested on a variety of sample types (tissue, blood, hair, feces) using fluorescent-labeled primers and an ABI 3730 automated DNA analysis system. The 5′ end of primer AMEL-F1 was fluorescently labeled with 6-FAM dye (blue) and the 5′ end of primer SRY-F1 was fluorescently labeled with HEX dye (green). These primers were used in a multiplex PCR reaction using a commercially-available multiplex kit (QIAgen®), along with unlabeled AMEL-R1 and SRY-R1 primers. The PCR mix for each sample included 5 μL of multiplex PCR mix (QIAgen®), 1 μL of a primer mix containing each of the four primers at 1 μM concentration, 2μL of ddH₂O, and 2 μL of undiluted DNA template (an estimated 50 to 300 ng). The cycling profile included an initial denaturation at 95° C. for 15 minutes, followed by 25-30 cycles of 94° C. for 30 seconds, 58° C. for 90 seconds, and 72° C. for 60 seconds with a final extension at 60° C. for 30 minutes. 0.25-1.0μL of the multiplex PCR product was then mixed with 8.85μL of Hi-Dye Formamide and 0.15 μL GeneScanSOO-ROX size standard and separated by capillary electrophoresis on an ABI 3730 DNA Analyzer. The raw array view and electropherogram results of this assay applied to 22 samples from 18 catarrhine genera are shown in FIG. 8. Shown in FIG. 9 is a portion of the ray array view for four DNA samples from two platyrrhine genera plus a negative control, as well as the corresponding electropherograms for the DNA samples (Gorilla and Papio fecal samples were also tested). All samples showed a strong blue (6-FAM label) fluorescent peak at just under 200 bp in size, indicating the presence of an X chromosome, and all known male samples displayed a smaller green (HEX label) band at just under 165 bp in size, indicating the presence of a Y chromosome.

Presented here is a combination of primers that can be used in a multiplex PCR assay to provide fast, reliable sex typing across the primate order.

In this assay, the positive PCR control amelogenin X fragment produced is, by design, somewhat larger than that of the SRY fragment and, hence, is the more likely amplicon to “drop out” when both target sequences are present. The assay thus allows identification of a sample as male if the SRY locus amplifies even in the absence of an X band. Nonetheless, in this study, nonamplification of the amelogenin X band in the presence of a Y band was observed regularly only in strepsirhines and only when using the original amelogenin primer pair (presumably due to mismatches between those primers and the target sequence) and was seen in anthropoids only occasionally, when hair was used as the source for template DNA. Additionally, minor modifications to the amelogenin primer set allowed amplification of a strepsirhine-specific X fragment, providing what appears to be an exactly comparable sex typing assay.

Although designed specifically to amplify the X chromosome copy of the amelogenin gene, the amelogenin primer pair is likely to simultaneously amplify the homologous Y chromosome copy of that gene in most primates, and in some taxa the Y homolog differs slightly in size from the X fragment. For example, the primers used here are known to flank a five base pair fixed sequence length variant, different from that used in the human sexing assay of Sullivan et al. (Sullivan et al., “A Rapid and Quantitative DNA Sex Test: Fluorescence-based PCR Analysis of X-Y Homologous Gene Amelogenin,” BioTechniques, 15:637-641 (1993), which is hereby incorporated by reference in its entirety) in several primates, including Homo, Pan, Pongo, and Papio, although not Saimiri (the one non-catarrhine for which the sequence of amelogenin Y has been published). It is noted that in FIG. 4A a second band, slightly larger than amelogenin X, can be seen in Homo and Pan males, while Callicebus appears to possess a slightly smaller putative amelogenin Y band. Similarly, in FIG. 8, Pan, Hylobates, Macaca, and Cercopithecus males show two amelogenin bands differing in size by ˜5 base pairs. It is thus likely that, at least for some taxa, the amelogenin primer pair presented here could be used on its own for sex typing as an alternative to those identified by Sullivan et al. (Sullivan et al., “A Rapid and Quantitative DNA Sex Test: Fluorescence-based PCR Analysis of X-Y Homologous Gene Amelogenin,” BioTechniques, 15:637-641 (1993), which is hereby incorporated by reference in its entirety).

The only other published molecular method for identifying the sex of a broad range of non-human primates outside of the hominoids (apes) appears to be effective only when high quality DNA samples are available. For example, Wilson and Erlandsson have designed a PCR-based sex typing assay applicable across anthropoid primates in which a single pair of primers is used to amplify homologous portions of the zinc finger protein gene from the nonrecombining regions of both the X and Y chromosomes (Wilson & Erlandsson, “Sexing of Human and Other Primate DNA,” Biol. Chem., 379:1287-1288 (1998), which is hereby incorporated by reference in its entirety). Like amelogenin, this region is characterized by a fixed sequence length difference between the X and Y copies of the gene, with the last intron of ZFX bearing a ˜400 base pair Alu insertion that predates the split between platyrrhines and catarrhines (see Shimmen et al., “Male-Driven Evolution of DNA Sequences,” Nature, 362:745-747 (1993), which is hereby incorporated by reference in its entirety). Unfortunately for most field molecular ecological studies, the X and Y fragments amplified in Wilson and Erlandsson's assay are, respectively, ˜700 and ˜1150 base pairs in length, generally beyond the size range that can be reliably amplified from degraded DNA samples. More recently, Fredsted and Villesen (Fredsted & Villesen, “Fast and Reliable Sexing of Prosimian and Human DNA,” Am. J Primatol., 64:345-350 (2004), which is hereby incorporated by reference in its entirety) have described a PCR-based sex-typing assay effective for strepsirhines and humans based on a ˜180 base pair sequence length difference on a different portion of the amelogenin gene X and Y copies. Again, however, at 1200 to 1400 base pairs in length, the target fragments for that assay are beyond the size range that can be reliably amplified from degraded DNA samples. In contrast, the present invention offers a set of primers that target for amplification much smaller regions (the largest being ˜200 base pairs), which are far more likely to be able to be amplified even from fragmented and degraded DNA extracted from noninvasively collected samples.

The multiplex PCR strategy described here is conceptually analogous to that used by Steiper and Ruvolo to sex type orangutans using a different set of amelogenin X and SRY primers originally designed for humans (Steiper & Ruvolo, “Genetic Sex Identification of Orangutans,” Anthropologischer Anzeiger, 61:1-5 (2003) (using amelogenin X primers reported in Sullivan et al., “A Rapid and Quantitative DNA Sex Test: Fluorescence-based PCR Analysis of X-Y Homologous Gene Amelogenin,” BioTechniques, 15:637-641 (1993) and SRY primers reported in Santos et al., “Reliability of DNA-Based Sex Tests,” Nature Genetics, 18:103 (1998)), all of which are hereby incorporated by reference in their entirety). However, although Steiper and Ruvolo did not test their primer combination outside of orangutans, it is unlikely that it would be broadly effective across primates, because of multiple mismatches between some of those primers and published nonhominoid amelogenin and SRY sequences. The multiplex PCR assay disclosed herein is also analogous to Pomp et al.'s multiplex method for sexing pig embryos (Pomp et al., “Sex Identification in Mammals With Polymerase Chain Reaction and Its Use to Examine Sex Effects on Diameter of Day-10 or -11 Pig Embryos,” J. Anim. Sci., 73:1408-1415 (1995), which is hereby incorporated by reference in its entirety). However, Pomp et al.'s X chromosome positive control fragment, located in ZFX, is around 450 base pairs and, hence, much more likely to fail to amplify from a degraded DNA sample. Finally, the procedure outlined here is also similar to that used by Taberlet et al. to sex type brown bears (Taberlet et al., “Sexing Free-Ranging Brown Bears Ursus arctos Using Hairs Found in the Field,” Mol. Ecol., 2:399-403 (1993), which is hereby incorporated by reference in its entirety) and that used by Murata and Masuda to sex type sloths using noninvasively collected hair samples (Murata & Masuda, “Gender Determination of the Linne's Two-Toed Sloth (Choloepus didactylus) Using SRYAmpified from Hair,” J. Vet. Med. Sci., 58:1157-1159 (1996), which is hereby incorporated by reference in its entirety), except that both of those methods amplify a small fragment of the mitochondrion rather than the X chromosome as a positive PCR control. The methods of the present invention are preferable to methods employing mitochondrial DNA as a control, because the number of X (control) and Y (test) templates in any given male sample should be exactly comparable since a nuclear template is used for both the control and the test. Contrastingly, the concentration of a mitochondrial template in a typical DNA extraction is expected to be several orders of magnitude greater than that of a nuclear template. Therefore, if a mitochondrial target is used as the positive PCR control, there is a much greater chance of amplifying only that control even if the Y target were present and, thus, mistakenly classifying a male sample as female.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method for identifying the sex of a primate, said method comprising:
providing a biological sample collected from said primate;
contacting the biological sample with one or more probes that hybridize to a target SRY nucleic acid molecule at one or more locations within a region spanning nucleotide 349 and nucleotide 513 of the nucleotide sequence of SEQ ID NO: 40 and/or its complement, under conditions effective to permit hybridization of the probes to any of the locations, if present, in the sample;
detecting any hybridization of the one or more probes to any of the locations; and
identifying the sex of the primate based on whether or not any hybridization occurs.

2. The method according to claim 1, wherein said contacting is carried out under polymerase chain reaction conditions.

3. The method according to claim 1, wherein the target SRY nucleic acid molecule is 150-300 nucleotides in length.

4. The method according to claim 1, wherein the probes comprise at least 45% of G+C bases.

5. The method according to claim 1, wherein the probes have an estimated melting temperature of at least 58° C.

6. The method according to claim 1, wherein each of the one or more probes has a greater hybridizing affinity for the target SRY nucleic acid molecule than their hybridizing affinity for the other one or more probes contacted with the sample.

7. The method according to claim 1, wherein the probes comprise the nucleotide sequence of SEQ ID NO: 32 and/or the nucleotide sequence of SEQ ID NO: 34.

8. The method according to claim 1 further comprising:
contacting the biological sample with one or more probes that hybridize to a target amelogenin nucleic acid molecule at one or more locations within a region spanning nucleotide 125 and nucleotide 323 of the nucleotide sequence of SEQ ID NO: 41 and/or its complement, and/or at one or more locations within a region spanning nucleotide 158 and nucleotide 350 of the nucleotide sequence of SEQ ID NO: 42 and/or its complement, under conditions effective to permit hybridization of the probes to any of the locations, if present, in the sample and
detecting any hybridization of the one or more probes that hybridize to a target amelogenin nucleic acid molecule.

9. The method according to claim 8, wherein the one or more probes comprise the nucleotide sequence of SEQ ID NO: 9, the nucleotide sequence of SEQ ID NO: 11, the nucleotide sequence of SEQ ID NO: 37, the nucleotide sequence of SEQ ID NO: 39, or combinations thereof.

10. The method according to claim 9, wherein the one or more probes comprise the nucleotide sequence of SEQ ID NO: 9 and/or the nucleotide sequence of SEQID 11.

11. The method according to claim 9, wherein the one or more probes comprise the nucleotide sequence of SEQ ID NO: 37 and/or the nucleotide sequence of SEQ ID NO: 39.

12. The method according to claim 8, wherein said contacting the biological sample with one or more probes that hybridize to a target amelogenin nucleic acid molecule is carried out under polymerase chain reaction conditions.

13. The method according to claim 12, wherein said contacting the biological sample with one or more probes that hybridize to a target SRY nucleic acid molecule is carried out under polymerase chain reaction conditions.

14. The method according to claim 13, wherein the region of the SRY nucleic acid molecule subjected to polymerase chain reaction conditions, if present, in the sample, is smaller than the region of the amelogenin nucleic acid molecule subjected to polymerase chain reaction conditions, if present, in the sample.

15. The method according to claim 8, wherein no hybridization between the one or more probes that hybridize to a target SRY nucleic acid molecule and the target SRY nucleic acid molecule, and hybridization between the one or more probes that hybridize to a target amelogenin nucleic acid molecule and the target amelogenin nucleic acid molecule, identifies the primate as a female.

16. The method according to claim 8, wherein hybridization between the one or more probes that hybridize to a target SRY nucleic acid molecule and the target SRY nucleic acid molecule, and hybridization between the one or more probes that hybridize to a target amelogenin nucleic acid molecule and the target amelogenin nucleic acid molecule, identifies the primate as a male.

17. The method according to claim 1, wherein at least one of the one or more locations within the region of the target SRY nucleic acid molecule is at least 20 nucleotides in length.

18. The method according to claim 1, wherein the one or more probes comprises a label to permit said detecting.

19. The method according to claim 18, wherein the label is selected from the group consisting of a fluorescent label, a radioactive label, a nuclear magnetic resonance active label, a bioluminescent label, and a chromophore label.

20. The method according to claim 1, wherein the primate is selected from the group consisting of Strepsirhini, Lemuroidea, Lemur, Lemur catta, Mirza coquereli, Daubentonia madagascariensis, Lorisoidea, Otolemur, Otolemur crassicaudatus, Otolemur garnetti, Haplorhini, Hominoidea, Homo, Homo sapiens, Pan, Pan troglodytes, Pongo, Pongo pygmaeus, Gorilla gorilla, Hylobates agilis, Symphalangus syndactylus, Cercopithecoidea, Cercopithecinae, Macaca sp., Cercopithecus sp., Cercopithecus ascanius, Cercopithecus nictitans, Cercocebus torquatus, Chlorocebus aethiops, Lophocebus aterrimus, Allenopithecus nigroviridis, Erythrocebus patas, Papio sp., Theropithecus gelada, Mandrillus leucophaeus, Mandrillus sphinx, Colobinae, Semnopithecus, Procolobus badius, Colobus guereza, Presbytis melalophos, Nasalis larvatus, Pygathrix nemaeus, Platyrrhini, Cebidae, Cebus albifrons, Saimiri, Saimiri sciureus, Aotus vociferans, Aotus lemurinus, Saguinus oedipus, Leontopithecus, Leontopithecus rosalia, Cebuella pygmaea, Pithecidae, Pithecia pithecia, Chiropotes satanus, Callicebus discolor, Callicebus donacophilus, Atelidae, Lagothrix, Lagothrix lagotricha, Ateles, Ateles belzebuth, Alouatta seniculus, Tarsioidea, Tarsiidae, and Tarsius syrichta.

21. The method according to claim 1, wherein the biological sample is selected from the group consisting of hair, feces, blood, tissue, urine, saliva, cheek cells, skin, and semen.

22. The method according to claim 1, wherein no hybridization between the one or more probes and the target SRY nucleic acid molecule identifies the primate as a female.

23. The method according to claim 1, wherein hybridization between the one or more probes and the target SRY nucleic acid molecule identifies the primate as a male.

24. A method for identifying the sex of a primate, said method comprising:
providing a biological sample collected from said primate;
contacting the biological sample with two or more different probes that hybridize to locations within a region of a target amelogenin nucleic acid molecule of an X chromosome and that hybridize to locations within a region of a target amelogenin nucleic acid molecule of a Y chromosome, under conditions effective to permit hybridization between the two or more probes and the locations, if present, in the sample, wherein the region of a target amelogenin nucleic acid molecule of the X chromosome and the region of a target amelogenin nucleic acid molecule of the Y chromosome have different lengths, and wherein the region of a target amelogenin nucleic acid molecule of the X chromosome spans nucleotide 125 and nucleotide 323 of the nucleotide sequence of SEQ ID NO: 41 and/or its complement;
detecting any hybridization of the two or more probes to any of the locations using polymerase chain reaction conditions; and
identifying the sex of the primate.

25. The method according to claim 24, wherein when the polymerase chain reaction yields a product of one length the primate is identified as a female.

26. The method according to claim 24, wherein when the polymerase chain reaction yields two homologous products of different length the primate is identified as a male.

27. The method according to claim 24, wherein the two or more probes comprise the nucleotide sequence of SEQ ID NO: 9 and SEQ ID NO: 11.
